(19) United States
(12) Patent Application Publication
(10) Pub. No.: US 2023/0183737 A1
(43) Pub. Date: Jun. 15, 2023

(54) TOMATO PLANTS HAVING SUPPRESSED MEIOTIC RECOMBINATION

(71) Applicant: NUNHEMS B.V., Nunhem (NL)

(72) Inventors: Wim VRIEZEN, Nunhem (NL); Hendrik Jacob SCHOUTEN, Wageningen (NL)

(21) Appl. No.: 17/924,321

(22) PCT Filed: May 7, 2021
(86) PCT No.: PCT/EP2021/062115

§ 371 Date: Nov. 9, 2022

(30) Foreign Application Priority Data
May 13, 2020 (EP) 20174476.0

(51) Int. Cl. C12N 15/82; C07K 14/415

(52) U.S. Cl. CPC C12N 15/8289; C07K 14/415; C12N 15/8212

(57) ABSTRACT

The present invention relates to a tomato plant comprising in its genome at least one chromosome comprising a mutant allele of the wild type male sterility 10 (MS10) gene and a mutant allele of the wild type anthocyanin absent (AA) gene wherein in said plant the meiotic recombination frequency is reduced between said mutant allele of the wild type MS10 gene and said mutant allele of the wild type AA gene when compared to the meiotic recombination frequency between the MS10 gene and the AA gene in a wild type Solanum lycopersicum plant. The present invention further relates to a seed from which a plant according to present invention can be grown and a part of a plant according to the present invention. The present invention further relates to a method of identifying and/or selecting a male sterile plant, said method comprising growing a plant according to the present invention and determining whether anthocyanin is absent in the hypocotyls of said plant. The present invention further relates to a method of identifying and/or selecting a plant or plant part according to the present invention. The present invention further relates to a method of producing tomato plant or tomato plant part having male sterility and anthocyanin absent hypocotyls, wherein in said plant or plant part the meiotic recombination frequency between the male sterility trait and the anthocyanin absent hypocotyls trait is reduced when compared to the meiotic recombination frequency between the MS10 gene and the AA gene in a wild type Solanum lycopersicum plant.

### FIELD OF THE INVENTION

The present invention relates to the field of plant breeding. Provided is a Solanum lycopersicum plant comprising in its genome at least one chromosome comprising a mutant allele of the wild type male sterility 10 (MS10) gene and a mutant allele of the wild type anthocyanin absent (AA) gene wherein in said plant the meiotic recombination frequency is reduced between said mutant allele of the wild type MS10 gene and said mutant allele of the wild type AA gene when compared to the meiotic recombination frequency between the MS10 gene and the AA gene in a wild type Solanum lycopersicum plant. The present invention further relates to a seed from which a plant according to present invention can be grown and a part of a plant according to the present invention. The present invention further relates to a method of identifying and/or selecting a male sterile plant, said method comprising growing a plant according to the present invention and determining whether anthocyanin is absent in the hypocotyls of said plant. The present invention further relates to a method of identifying and/or selecting a plant or plant part according to the present invention. The present invention further relates to a method of producing tomato plant or tomato plant part having male sterility and anthocyanin absent hypocotyls, wherein in said plant or plant part the meiotic recombination frequency between the male sterility trait and the anthocyanin absent hypocotyls trait is reduced when compared to the meiotic recombination frequency between the MS10 gene and the AA gene in a wild type Solanum lycopersicum plant.

### BACKGROUND

In commercial tomato production predominantly F1 hybrid Solanum lycopersicum varieties are cultivated since such varieties provide a high yield in combination with other superior quality characteristics such as plant architecture, disease resistance and fruit quality. The production of F1 hybrid tomato seeds requires that the female parent does not produce functional anthers, pollen, or male gametes. Conventionally, this is achieved by manually emasculating all flowers of the female parent which is very laborious and costly. Alternatively, a male sterility (MS) system may be used wherein such manual emasculation of the flowers is not necessary. Using a MS system furthermore prevents contamination of hybrid seeds from accidentally self-pollinated flowers.

A known MS system in Solanum lycopersicum is based on the single recessive male sterile 10 (MS10) gene and which has been used for decades for tomato F1 hybrid seed production; see Kumar & Singh (2005) Mechanisms for hybrid development in vegetables. Journal of New Seeds 6, 381-407. Solanum lycopersicum plants that are homozygous for a mutant ms10 allele show complete male sterility in combination with normally developed pistils that are accessible for hand pollination to produce F1 hybrid seeds. The MS10 gene has been described to encode a basic helix-loop-helix transcription factor. Mutations resulting in no expression or sufficiently reduced expression of the wild type MS10 gene and/or resulting in the expression of a mutant ms10 protein having loss-of-function or sufficiently reduced function when compared to the wild type MS10 protein lead to the male sterility phenotype.

The female ms10 plants required for F1 hybrid Solanum lycopersicum seeds are obtained by back-crossing or selfing plants that are heterozygous for the used mutant ms10 allele. Such back-crossing or selfing results in offspring which is a mixture of male sterile plants homozygous for the mutant ms10 allele and plants that are not homozygous for the mutant ms10 allele that are not useful for F1 hybrid seed production. The male sterile plants that are homozygous for the mutant ms10 allele can be distinguished from the remaining progeny plants by marker assisted selection, which, however, is time-consuming and relatively expensive.

Alternatively, it has been described that the locus of the MS10 gene on chromosome 2 of the Solanum lycopersicum genome is relatively close to the locus of the anthocyanin absent (AA) gene (Zhang et al. Mol Breeding (2016) 36:107). Seedlings of tomato plants that are homozygous for a mutant allele of the AA gene can be visually distinguished from heterozygous seedlings and seedlings homozygous for the wild type allele by their green colour of the hypocotyl. It has subsequently been suggested that by using tomato plants having both a mutant AA gene and a mutant MS10 gene on chromosome 2 of the AA gene, male sterile plants that are homozygous for the mutant ms10 allele can be visually selected based on hypocotyl colour. However, loci of the MS10 gene and the AA gene on chromosome 2 are about 1.2 Mbp apart, which corresponds to genetic distance of approximately 7 cM. This means that in about 7% of the gametes produced by a heterozygous plant the mutant aa allele is no longer coupled to the mutant ms10 allele. Consequently, it is not possible to reliably select the male sterile plants solely based on the colour of the seedling's hypocotyl as this would result in the selection of parental plants which show the anthocyanin absent phenotype of the hypocotyl but that do not have the male sterile phenotype. Accordingly selected parental plants that show the anthocyanin absent phenotype of the hypocotyl still need to be subjected to marker assisted selection to prevent that the hybrid seeds are contaminated due to accidental self-pollination. There is thus a need for a MS system in tomato breeding, which allows a more reliable, time- and cost-efficient selection of male sterile plants based on the colour of the seedling's hypocotyl.

### SUMMARY OF THE INVENTION

The present invention provides a plant of the species Solanum lycopersicum comprising in its genome at least one chromosome comprising a mutant allele of the wild type male sterility 10 (MS10) gene and a mutant allele of the wild type anthocyanin absent (AA) gene wherein in said plant the meiotic recombination frequency is reduced between said mutant allele of the wild type MS10 gene and said mutant allele of the wild type AA gene when compared to the meiotic recombination frequency between the MS10 gene and the AA gene in a wild type Solanum lycopersicum plant, wherein the wild type MS10 gene encodes a protein comprising at least 95% amino acid sequence identity to SEQ ID NO: 1 and the mutant allele of the wild type MS10 gene results in no expression or reduced expression of the wild type gene and/or the mutant allele of the wild type MS10 gene encodes a protein having loss-of-function or reduced function when compared to the wild type protein, and wherein the wild type AA gene encodes a protein comprising at least 95% amino acid sequence identity to SEQ ID NO: 3 and the mutant allele of the wild type AA gene results in no expression or reduced expression of the wild type gene and/or the mutant allele of the wild type AA gene encodes a protein having loss-of-function or reduced function when compared to the wild type protein.

The present invention further relates to a seed from which a plant according to present invention can be grown and a part of a plant according to the present invention, wherein said plant part preferably is a leaf, anther, pistil, stem, petiole, root, ovule, pollen, microspore, protoplast, callus, tissue, seed, flower, cotyledon, hypocotyl, embryo or cell.

Also provided herein is a part of the plant according to the present invention, wherein said plant part preferably is a leaf, anther, pistil, stem, petiole, root, ovule, pollen, microspore, protoplast, callus, tissue, seed, flower, cotyledon, hypocotyl, embryo or cell.

Further provided herein is a method of identifying and/or selecting a male sterile plant, said method comprising growing a plant according to the present invention and determining whether anthocyanin is absent in the hypocotyls of said plant.

Further provided herein is a method of identifying and/or selecting a plant or plant part of the species Solanum lycopersicum comprising in its genome at least one chromosome comprising a mutant allele of the wild type MS10 gene and a mutant allele of the wild type AA gene, wherein the wild type MS10 gene encodes a protein comprising at least 95% amino acid sequence identity to SEQ ID NO: 1 and the mutant allele of the wild type MS10 gene results in no expression or reduced expression of the wild type gene and/or the mutant allele of the wild type MS10 gene encodes a protein having loss-of-function or reduced function when compared to the wild type protein, and wherein the wild type AA gene encodes a protein comprising at least 95% amino acid sequence identity to SEQ ID NO: 3 and the mutant allele of the wild type AA gene results in no expression or reduced expression of the wild type gene and/or the mutant allele of the wild type AA gene encodes a protein having loss-of-function or reduced function when compared to the wild type protein, and wherein said method comprises determining whether in the plant or plant part the meiotic recombination frequency between the MS10 gene and the AA gene is reduced gene when compared to the meiotic recombination frequency between the MS10 gene and the AA gene in a wild type Solanum lycopersicum plan.

Further provided herein is a method for producing plant or plant part of the species Solanum lycopersicum having male sterility and anthocyanin absent hypocotyls, wherein in said plant or plant part the meiotic recombination frequency between the male sterility trait and the anthocyanin absent hypocotyls trait is reduced when compared to the meiotic recombination frequency between the MS10 gene and the AA gene in a wild type Solanum lycopersicum plant, said method comprising: (a) inducing in a plant or plant part a double strand break in both the MS10 gene and the AA gene, wherein the wild type MS10 gene encodes a protein comprising at least 95% amino acid sequence identity to SEQ ID NO: 1 and wherein the wild type AA gene encodes a protein comprising at least 95% amino acid sequence identity to SEQ ID NO: 3; (b) optionally regenerating the plant part in which the double strand break is induced into a plant or into a different plant part.

### BRIEF DESCRIPTION OF THE FIGURES

FIG. 1: Overview of the approach of Example 1. A. Schematic picture of the positions of the involved genes on Chromosome 2 of tomato. MS refers the male sterility gene, and AA to the anthocyanin absence gene. The sizes and positions of the genes are not drawn to scale. B. The positions of the CRISPR-Cas induced double strand breaks in these two genes are shown as lighting flashes. A small proportion of the excised chromosomal fragments are repaired in the opposite orientation, leading to an induced inversion. Moreover, both genes (MS and AA) are truncated and lose their function. This leads to male sterility and anthocyanin absence. In hybrids the genetic distance between ms and aa is reduced to about 0 cM, because of recombination suppression by the induced inversion. C. The primers for checking presence of induced inversions are shown as small arrows.

FIG. 2: Representation of a targeted induced inversion. The used CRISPR-Cas9 constructs contained two gRNAs per construct, such as gMS1 and gAA1, targeting the MS-gene and the AA-gene, respectively. When a double-strand break was induced at both sites in the same chromosome, inversion of the DNA fragment in between could occur. An inversion will lead to the inactivation of both genes since part of one gene is inversely fused to the remaining part of the other gene. Well-designed PCR-primers were used to unambiguously detect inversion-events. In this example, two primer-sites on the same DNA strand at the borders of the inversion (MS-R and AA-R) are oriented in the wild type genome in a manner that prevents any amplification in a PCR. However, after inversion the new locations of the primer binding sites are close together and in opposite directions, which makes amplification of a DNA fragment possible.

FIG. 3: The upper part of the figures represents the wild type reference genome. After inversion, the sequence at the gMS side is inverted and linked to the gAA side with is depicted by the arrows. The gRNA sequences, which were ˜1.1 Mbp apart on the reference genome, were linked together as shown by the sequence at the bottom. The alignment in the lower part of the figure shows that the DNA has been cleaved at the predicted location of the gRNA binding sites. The fraction of the gRNA sequence in bold corresponds to the sequence at the other side of the inversion. DNA sequence analysis of one end of an induced inversion after transfection with construct 1. The Sanger sequence is part of the PCR product generated with primers MS-R and AA-R and genomic DNA from protoplasts transfected with construct 1 containing the gRNA sequences gMS1 and gAA1. The Sanger sequence refers to the right-hand side of the inversion, and the flanking DNA at that side.

FIG. 4: The upper part of the figures represents the wild type reference genome. After inversion, the sequence at the gMS side is inverted and linked to the gAA side with is depicted by the arrows. The gRNA sequences, which were ˜1.1 Mbp apart on the reference genome, were linked together as shown by the sequence at the bottom. The alignment in the lower part of the figure shows that the DNA has been cleaved at the predicted location of the gRNA binding sites. The fraction of the gRNA sequence in bold corresponds to the sequence at the other side of the inversion. DNA sequence analysis of one end of an induced inversion after transfection with construct 3. The Sanger sequence is part of the PCR product generated with primers MS-F and AA-F and genomic DNA from protoplasts transfected with construct 3 containing the gRNA sequences gMS3 and gAA3. The Sanger sequence refers to the left-hand side of the inversion, and the flanking DNA at that side.

FIG. 5: The upper part of the figures represents the wild type reference genome. After inversion, the sequence at the gMS side is inverted and linked to the gAA side with is depicted by the arrows. The gRNA sequences, which were ˜1.1 Mbp apart on the reference genome, were linked together as shown by the sequence at the bottom. The alignment in the lower part of the figure shows that the DNA has been cleaved at the predicted location of the gRNA binding sites. The fraction of the gRNA sequence in bold corresponds to the sequence at the other side of the inversion. DNA sequence analysis of one end of an induced inversion after transfection with construct 4. The Sanger sequence is part of the PCR product generated with primers MS-R and AA-R and genomic DNA from protoplasts transfected with construct 4 containing the gRNA sequences gMS4 and gAA4. The Sanger sequence refers to the right-hand side of the inversion, and the flanking DNA at that side.

### DETAILED DESCRIPTION OF THE INVENTION

### General Definitions

The term “genome” relates to the genetic material of an organism. It consists of DNA. The genome includes both the genes and the non-coding sequences of the DNA.

An allelism test is a test known in the art that can be used to identify whether two genes conferring the same trait are located at the same locus.

The term “genetic determinant” relates to the genetic information in the genome of the plant that causes a particular trait of a plant. Accordingly, a genetic determinant comprises the genetic information (gene or locus or introgression) that confers a certain trait. In general, a genetic determinant may comprise a single gene (or one Quantitative Trait Locus (QTL)) or more than one gene. In the present invention, the genetic determinant for the male sterility 10 trait comprises a single gene. Furthermore, the genetic determinant for anthocyanin absent trait comprises a single gene.

The word “trait” in the context of this application refers to the phenotype of the plant. When a plant shows either one or both traits of the invention, its genome comprises either one or both mutant alleles causing the traits of the invention, particularly in the present invention when said either one or both mutant alleles are in homozygous form. It is understood that when referring to a plant comprising both traits of the invention, reference is made to a tomato plant comprising the male sterility 10 trait and the anthocyanin absent trait as further described herein.

A genetic determinant can be inherited in a recessive manner, an intermediate manner, or in a dominant manner. Selection for the phenotypic trait is easier when intermediate or dominant inheritance is involved, as a larger part of the progeny of a cross reveals the trait. In general, a genetic determinant can also comprise a combination of recessive and/or intermediate and/or dominant genes or QTLs. In the present invention, the genetic determinant for the male sterility 10 trait comprises a single recessive gene. Furthermore, the genetic determinant for anthocyanin absent trait comprises a single recessive gene.

Selection for a genetic determinant (e.g. the mutant ms10 allele and/or the mutant aa allele) can be done on phenotype (the trait that can be observed). Selection can also be done by using molecular genotyping methods, such as one or more molecular markers that are genetically linked to the mutant allele or preferably using the gene or allele sequence itself, e.g. by molecular methods which are able to distinguish between the presence of a mutant allele and wild type allele, or products thereof (such as mRNA or protein encoded by the allele). The use of molecular genotyping methods in breeding (such as “marker assisted selection” when genetically linked markers are used, or other genotyping methods, such as SNP genotyping) requires a smaller population for screening (when compared to phenotypical selection) and can be done in a very early stage. A further advantage of molecular genotyping methods is the possibility to easily distinguish between homozygous plants or seeds having no wild type copies of the MS10 gene (homozygous for the mutant ms10 allele) and/or having no wild type copies of the AA gene (homozygous for the mutant aa allele), heterozygous plants or seeds having one wild type copy and one mutant copy of the MS10 gene (heterozygous for the mutant ms10 allele) and/or having one wild type copy and one mutant copy of the AA gene (heterozygous for the mutant aa allele) and homozygous plants or seeds having no copies of the mutant MS10 gene and/or having no copies of the mutant AA gene of the present invention, which can be done even before seeds germinate or in early plant development, e.g. before mature flowers have developed.

A “plant line” or “breeding line” refers to a plant and its progeny. As used herein, the term “inbred line” refers to a plant line which has been repeatedly selfed and is nearly homozygous for every characteristic. Thus, an “inbred line” or “parent line” refers to a plant which has undergone several generations (e.g. at least 5, 6, 7 or more) of inbreeding, resulting in a plant line with a high uniformity.

The term “allele(s)” means any of one or more alternative forms of a gene at a particular locus, all of which alleles relate to one trait or characteristic at a specific locus. In a diploid cell of an organism, alleles of a given gene are located at a specific location, or locus (loci plural) on a chromosome. One allele is present on each chromosome of the pair of homologous chromosomes. A diploid plant species may comprise a large number of different alleles at a particular locus. These may be identical alleles of the gene (homozygous) or two different alleles (heterozygous).

The term “locus” (plural loci) means a specific place or places or a site on a chromosome where for example a gene or genetic marker is found. The male sterility 10 locus (or loci) of the present invention thus is the location in the genome of a tomato plant where the MS10 gene is found. The anthocyanin absent locus (or loci) of the present invention thus is the location in the genome of a tomato plant where the AA gene is found.

The term “gene” means a (genomic) DNA sequence comprising a region (transcribed region), which is transcribed into a messenger RNA molecule (mRNA) in a cell, and an operably linked regulatory region (also described herein as regulatory sequence, e.g. a promoter). A gene may thus comprise several operably linked sequences, such as a promoter, a 5′ leader sequence comprising e.g. sequences involved in translation initiation, a (protein) coding region (cDNA or genomic DNA) and a 3′ non-translated sequence comprising e.g. transcription termination sites. Different alleles of a gene are thus different alternative forms of the gene, which may be in the form of e.g. differences in one or more nucleotides of the genomic DNA sequence (e.g. in the promoter sequence, the exon sequences, intron sequences, etc.), mRNA and/or amino acid sequence of the encoded protein. A gene may be an endogenous gene (in the species of origin) or a chimeric gene (e.g. a transgene or cis-gene). The “promoter” of a gene sequence is defined as a region of DNA that initiates transcription of a particular gene. Promoters are located near the genes they transcribe, on the same strand and upstream on the DNA. Promoters can be about 100-1000 base pairs long. In one aspect the promoter is defined as the region of about 1000 base pairs or more e.g. about 1500 or 2000, upstream of the start codon (i.e. ATG) of the protein encoded by the gene.

“Transgene” or “chimeric gene” refers to a genetic locus comprising a DNA sequence, such as a recombinant gene, which has been introduced into the genome of a plant by transformation, such as Agrobacterium mediated transformation. A plant comprising a transgene stably integrated into its genome is referred to as “transgenic plant”.

“Expression of a gene” refers to the process wherein a DNA region, which is operably linked to appropriate regulatory regions, particularly a promoter, is transcribed into an RNA, which is biologically active, i.e. which is capable of being translated into a biologically active protein or peptide (or active peptide fragment) or which is active itself (e.g. in posttranscriptional gene silencing or RNAi). The coding sequence may be in sense-orientation and encodes a desired, biologically active protein or peptide, or an active peptide fragment.

A “quantitative trait locus”, or “QTL” is a chromosomal locus that encodes for one or more alleles that affect the expressivity of a continuously distributed (quantitative) phenotype.

“Physical distance” between loci (e.g. between genes and/or between molecular markers and/or between phenotypic markers) on the same chromosome is the actual physical distance expressed in bases or base pairs (bp), kilo bases or kilo base pairs (kb) or megabases or mega base pairs (Mb).

“Genetic distance” between loci (e.g. between molecular markers and/or between phenotypic markers) on the same chromosome is measured by frequency of crossing-over, or recombination frequency (RF) and is indicated in centimorgans (cM). One cM corresponds to a recombination frequency of 1%. If no recombinants can be found, the RF is zero and the loci are either extremely close together physically or they are identical. The further apart two loci are, the higher the RF.

“Wild type allele” (WT) refers herein to a version of a gene encoding a fully functional protein (wild type protein).

Accordingly, the term “wild type MS10 allele” or “MS10 allele” or “wild type allele of the MS10 gene” refers to the fully functional allele of the MS10 gene, which allows normal protein function (i.e. normal protein expression in combination with normal enzymatic activity of the expressed protein) when compared to a wild type MS10 allele. The MS10 gene encodes a basic helix-loop-helix transcription factor. One example of a wild type MS10 allele in the species Solanum lycopersicum for instance is the wild type genomic DNA which encodes the wild type MS10 cDNA (mRNA) sequence depicted in SEQ ID NO:2. The protein sequence encoded by this wild type MS10 cDNA has 209 amino acid residues and is depicted in SEQ ID NO:1, which corresponds to NCBI reference sequence XM_026029418.1. The wild type Solanum lycopersicum MS10 allele further comprises functional variants of the wild type genomic DNA which encodes the wild type MS10 cDNA and amino acid sequences as described herein. Whether a certain variant of the herein specifically described wild type MS10 allele represents a “functional variant” can be determined by using routine methods, including, but not limited to phenotypic testing for normal viable pollen production and in silico prediction of amino acid changes that affect protein function. For instance, a web-based computer program SIFT (Sorting Intolerant from Tolerant) is a program that predicts whether an amino acid substitution affects protein function; see world wide web at sift.bii.a-star.edu.sg/. Functionally important amino acids will be conserved in the protein family, and so changes at well-conserved positions tend to be predicted as not tolerated or deleterious; see also Ng and Henikoff (2003) Nucleic Acids Res 31(13): 3812-3814. For example, if a position in an alignment of a protein family only contains the amino acid isoleucine, it is presumed that substitution to any other amino acid is selected against and that isoleucine is necessary for protein function. Therefore, a change to any other amino acid will be predicted to be deleterious to protein function. If a position in an alignment contains the hydrophobic amino acids isoleucine, valine and leucine, then SIFT assumes, in effect, that this position can only contain amino acids with hydrophobic character. At this position, changes to other hydrophobic amino acids are usually predicted to be tolerated but changes to other residues (such as charged or polar) will be predicted to affect protein function. An alternative tool useful for the prediction of protein function is Provean; see world wide web at provean.jcvi.org/index.php. Also, an ortholog of the Solanum lycopersicum MS10 gene, particularly in a wild relative of the species Solanum lycopersicum, may be a functional variant of the wild type MS10 allele provided that said variant allows normal protein function.

The term “wild type AA allele” or “AA allele” or “wild type allele of the AA gene” refers to the fully functional allele of the AA gene, which allows normal protein function (i.e. normal protein expression in combination with normal enzymatic activity of the expressed protein) when compared to a wild type AA allele. The AA gene encodes a glutathione S-transferase enzyme. One example of a wild type AA allele in the species Solanum lycopersicum for instance is the wild type genomic DNA which encodes the wild type AA cDNA (mRNA) sequence depicted in SEQ ID NO:4. The protein sequence encoded by this wild type AA cDNA has 230 amino acid residues and is depicted in SEQ ID NO:3, which corresponds to NCBI reference sequence XM_004232621.4. The wild type Solanum lycopersicum AA allele further comprises functional variants of the wild type genomic DNA which encodes the wild type AA cDNA and amino acid sequences as described herein. Whether a certain variant of the herein specifically described wild type AA allele represents a “functional variant” can be determined by using routine methods, including, but not limited to testing of enzymatic activity, phenotypic testing for hypocotyl colour and in silico prediction of amino acid changes that affect protein function as further described herein above. Also, an ortholog of the Solanum lycopersicum AA gene, particularly in a wild relative of the species Solanum lycopersicum, may be a functional variant of the wild type AA allele provided that said variant allows normal protein function.

“Mutant allele” refers herein to an allele comprising one or more mutations when compared to the wild type allele, resulting in the trait of the present invention. The one or more mutations may be in the coding sequence (mRNA, cDNA or genomic sequence) or in the associated non-coding sequence and/or regulatory sequence regulating the level of expression of the coding sequence. Such mutation(s) (e.g. insertion, inversion, deletion and/or replacement of one or more nucleotide(s)) may lead to the encoded protein having reduced in vitro and/or in vivo functionality (reduced function) or no in vitro and/or in vivo functionality (loss-of-function), e.g. due to the protein being truncated or having an amino acid sequence wherein one or more amino acids are deleted, inserted or replaced. Such changes may lead to the protein having a different 3D conformation, being targeted to a different sub-cellular compartment, having one or more modified catalytic domains, having a modified binding activity to nucleic acids or proteins, etc. preferably, the mutant allele of the present invention encodes a truncated protein having decreased function or loss-of-function when compared to the wild type protein. Furthermore, the mutation(s) (e.g. insertion, inversion, deletion and/or replacement of one or more nucleotide(s)) may lead to the encoded protein having reduced expression or no protein expression.

Accordingly, the term “mutant ms10 allele” or “ms10 allele” or “mutant allele of the MS10 gene” or “mutant allele of the wild type MS10 gene” inter alia refers to an allele of the MS10 gene comprising one or more mutations in the coding sequence, which one or more mutations leads to a reduced function or loss-of-function of encoded gene product and which causes the plants to have the male sterility trait when the mutant allele is in homozygous form. The term “male sterility” or “male sterility trait” refers to a plant trait which results in the failure of the plant to produce functional anthers, pollen, or male gametes. The term mutant ms10 allele also comprises knock-out ms10 alleles and knock-down ms10 alleles, as well as ms10 alleles encoding a mutant ms10 protein having reduced function or no function. As used herein, the term “knockout allele” refers to an allele wherein the expression of the respective (wild type) gene is not detectable anymore. A “knock-down” allele has reduced expression of the respective (wild type) gene compared to the wild type allele.

Accordingly, the term “mutant aa allele” or “aa allele” or “mutant allele of the AA gene” or “mutant allele of the wild type AA gene” inter alia refers to an allele of the AA gene comprising one or more mutations in the coding sequence, which one or more mutations leads to a reduced function or loss-of-function of encoded gene product and which causes the plants to have the anthocyanin absent trait when the mutant allele is in homozygous form. The term “anthocyanin absent” or “anthocyanin absent trait” refers to a plant trait which results in the absence of anthocyanin coloration in the hypocotyls of said plant. The term mutant aa allele also comprises knock-out aa alleles and knock-down aa alleles, as well as aa alleles encoding a mutant aa protein having reduced function or no function.

The term “induced mutant allele” as used herein refers to any allele of the wild type gene resulting in the trait of the present invention which is produced by human intervention, such as mutagenesis. Preferably, the induced mutant allele cannot be found in plants in the natural population or breeding population.

The term “natural mutant allele” as used herein refers to any allele of the wild type gene resulting in the trait of the present invention wherein the mutant allele evolved without direct human intervention. Preferably, the natural mutant allele can be found in plants in the natural population or breeding population.

“Wild type plant” refers herein to a tomato plant, preferably a plant of the species Solanum lycopersicum, comprising two copies of the wild type MS10 allele and/or two copies of the wild type AA allele and thus is considered to show normal viable pollen production and normal coloration of the hypocotyl. Such plants are for example suitable controls in phenotypic essays, particularly if said control plants have the same genetic background as the plants (e.g. mutant plants) that are subjected to phenotypic testing.

In a tomato plant the wild type MS10 gene encodes a protein comprising at least 95% (96%, 97%, 98%, 98.3%, 98.7%, 99.0%, or 99.3% or more preferably 99.7%) amino acid sequence identity to SEQ ID NO:1. The protein described by the amino acid sequence SEQ ID NO:1 represents the wild type MS10 protein in Solanum lycopersicum and corresponds to NCBI reference sequence XM_026029418.1. In wild relatives of Solanum lycopersicum the wild type MS10 protein accordingly is encoded by an ortholog of the wild type MS10 gene in Solanum lycopersicum. Preferably, the ortholog of the Solanum lycopersicum MS10 gene in wild relatives of Solanum lycopersicum encodes a protein having at least 95% (e.g. at least 96%, 97%, 98%, 98.3%, 98.7%, 99.0%, or 99.3% or more preferably 99.7%) amino acid sequence identity to SEQ ID NO:1.

In a tomato plant the wild type AA gene encodes a protein comprising at least 95% (96%, 97%, 98%, 98.3%, 98.7%, 99.0%, or 99.3% or more preferably 99.7%) amino acid sequence identity to SEQ ID NO:3. The protein described by the amino acid sequence SEQ ID NO:3 represents the wild type AA protein in Solanum lycopersicum and corresponds to NCBI reference sequence XM_004232621.4. In wild relatives of Solanum lycopersicum the wild type AA protein accordingly is encoded by an ortholog of the wild type AA gene in Solanum lycopersicum. Preferably, the ortholog of the Solanum lycopersicum AA gene in wild relatives of Solanum lycopersicum encodes a protein having at least 95% (e.g. at least 96%, 97%, 98%, 98.3%, 98.7%, 99.0%, or 99.3% or more preferably 99.7%) amino acid sequence identity to SEQ ID NO:3.

The term “orthologous gene” or “ortholog” is defined as genes in different species that have evolved through speciation events. It is generally assumed that orthologs have the same biological functions in different species. Accordingly, it is particularly preferred that the protein encoded by the ortholog of the wild type Solanum lycopersicum MS10 gene in wild relatives of the species Solanum lycopersicum has the same biological function as the wild type Solanum lycopersicum MS10 protein. Furthermore, it is particularly preferred that the protein encoded by the ortholog of the wild type Solanum lycopersicum AA gene in wild relatives of the species Solanum lycopersicum has the same biological function as the wild type Solanum lycopersicum AA protein. Methods for the identification of orthologs is very well known in the art as it accomplishes two goals: delineating the genealogy of genes to investigate the forces and mechanisms of evolutionary process and creating groups of genes with the same biological functions (Fang G, et al (2010) Getting Started in Gene Orthology and Functional Analysis. PLoS Comput Biol 6(3): e1000703. doi:10.1371/journal.pcbi.1000703). For instance, orthologs of a specific gene or protein can be identified using sequence alignment or sequence identity of the gene sequence of the protein of interest with gene sequences of other species. Gene alignments or gene sequence identity determinations can be done according to methods known in the art, e.g. by identifying nucleic acid or protein sequences in existing nucleic acid or protein database (e.g. GENBANK, SWISSPROT, TrEMBL) and using standard sequence analysis software, such as sequence similarity search tools (BLASTN, BLASTP, BLASTX, TBLAST, FASTA, etc.). In one aspect of the invention an ortholog of the Solanum lycopersicum MS10 protein in wild relatives of Solanum lycopersicum has at least 95% (e.g. at least 96%, 97%, 98%, 98.3%, 98.7%, 99.0%, or 99.3% or more preferably 99.7%) amino acid sequence identity with SEQ ID NO: 1. In one aspect of the invention an ortholog of the Solanum lycopersicum AA protein in wild relatives of Solanum lycopersicum has at least 95% (e.g. at least 96%, 97%, 98%, 98.3%, 98.7%, 99.0%, or 99.3% or more preferably 99.7%) amino acid sequence identity with SEQ ID NO: 3.

“Introgression fragment” or “introgression segment” or “introgression region” refers to a chromosome fragment (or chromosome part or region) which has been introduced into another plant of the same or related species by crossing or traditional breeding techniques, such as backcrossing, i.e. the introgressed fragment is the result of breeding methods referred to by the verb “to introgress” (such as backcrossing). It is understood that the term “introgression fragment” never includes a whole chromosome, but only a part of a chromosome. The introgression fragment can be large, e.g. even three-quarters or half of a chromosome, but is preferably smaller, such as about 15 Mb or less, such as about 10 Mb or less, about 9 Mb or less, about 8 Mb or less, about 7 Mb or less, about 6 Mb or less, about 5 Mb or less, about 4 Mb or less, about 3 Mb or less, about 2.5 Mb or 2 Mb or less, about 1 Mb (equals 1,000,000 base pairs) or less, or about 0.5 Mb (equals 500,000 base pairs) or less, such as about 200,000 bp (equals 200 kilo base pairs) or less, about 100,000 bp (100 kb) or less, about 50,000 bp (50 kb) or less, about 25,000 bp (25 kb) or less.

The term “isogenic plant” refers to two plants which are genetically identical except for the mutant allele of the present invention. In order to investigate the impact of the male sterility trait and/or the anthocyanin absent trait as described herein, one can cross a plant line (or variety) of interest with a plant comprising the mutant allele causing the male sterility trait and/or the mutant allele causing the anthocyanin absent trait and select for progeny expressing the desired trait. Optionally one may have to self the progeny one or more times to be able to determine the genetic determinants for the male sterility trait and/or the anthocyanin absent trait in the plant phenotype. Said progeny can then be backcrossed (at least 2 times, e.g. 3, 4, or preferably 5 or 6 times) with the plant line (or variety) of interest while selecting for progeny having the same phenotype as the plant line (or variety) of interest and expressing the genetic determinants for the male sterility trait and/or the anthocyanin absent trait. The impact of the mutant allele causing the male sterility trait and/or the anthocyanin absent trait can then be compared between the plant line (variety) of interest and its isogenic line not comprising the genetic determinants for the male sterility trait and/or the anthocyanin absent trait.

The term “nucleic acid sequence” or “nucleic acid molecule” or polynucleotide are used interchangeably and refer to a DNA or RNA molecule in single or double stranded form, particularly a DNA encoding a protein or protein fragment according to the invention. An “isolated nucleic acid sequence” refers to a nucleic acid sequence which is no longer in the natural environment from which it was isolated, e.g. the nucleic acid sequence in a bacterial host cell or in the plant nuclear or plastid genome.

The terms “protein”, “peptide sequence”, “amino acid sequence” or “polypeptide” are used interchangeably and refer to molecules consisting of a chain of amino acids, without reference to a specific mode of action, size, 3-dimensional structure or origin. A “fragment” or “portion” of a protein may thus still be referred to as a “protein”. An “isolated protein” is used to refer to a protein which is no longer in its natural environment, for example in vitro or in a recombinant bacterial or plant host cell.

An “active protein” or “functional protein” is a protein which has protein activity as measurable in vitro, e.g. by an in vitro activity assay, and/or in vivo, e.g. by the phenotype conferred by the protein. A “wild type” protein is a fully functional protein, as present in the wild type plant. A “mutant protein” is herein a protein comprising one or more mutations in the nucleic acid sequence encoding the protein, whereby the mutation results in (the mutant nucleic acid molecule encoding) a protein having altered activity, preferably a protein having reduced activity, most preferably a protein having no activity.

“Functional derivatives” of a protein as described herein are fragments, variants, analogues, or chemical derivatives of the protein which retain at least a portion of the activity or immunological cross reactivity with an antibody specific for the mutant protein.

A fragment of a mutant protein refers to any subset of the molecule.

Variant peptides may be made by direct chemical synthesis, for example, using methods well known in the art.

An analogue of a mutant protein refers to a non-natural protein substantially similar to either the entire protein or a fragment thereof.

A “mutation” in a nucleic acid molecule is a change of one or more nucleotides compared to the wild type sequence, e.g. by replacement, deletion, inversion or insertion of one or more nucleotides.

A “mutation” in an amino acid molecule making up a protein is a change of one or more amino acids compared to the wild type sequence, e.g. by replacement, deletion or insertion of one or more amino acids. Such a protein is then also referred to as a “mutant protein”.

A “point mutation” is the replacement of a single nucleotide, or the insertion or deletion of a single nucleotide.

A “nonsense mutation” is a (point) mutation in a nucleic acid sequence encoding a protein, whereby a codon in a nucleic acid molecule is changed into a stop codon. This results in a pre-mature stop codon being present in the mRNA and results in translation of a truncated protein. A truncated protein may have decreased function or loss of function.

A “missense or non-synonymous mutation” is a (point) mutation in a nucleic acid sequence encoding a protein, whereby a codon is changed to code for a different amino acid. The resulting protein may have decreased function or loss of function.

A “splice-site mutation” is a mutation in a nucleic acid sequence encoding a protein, whereby RNA splicing of the pre-mRNA is changed, resulting in an mRNA having a different nucleotide sequence and a protein having a different amino acid sequence than the wild type. The resulting protein may have decreased function or loss of function.

A “frame shift mutation” is a mutation in a nucleic acid sequence encoding a protein by which the reading frame of the mRNA is changed, resulting in a different amino acid sequence. The resulting protein may have decreased function or loss of function.

A “deletion” in context of the invention shall mean that anywhere in a given nucleic acid sequence at least one nucleotide is missing compared to the nucleic sequence of the corresponding wild type sequence or anywhere in a given amino acid sequence at least one amino acid is missing compared to the amino acid sequence of the corresponding (wild type) sequence.

An “inversion” in context of the invention shall mean a mutation wherein in a given nucleic acid sequence the nucleotide sequence of a fragment of at least 3 or more nucleotides is reversed when compared to the wild type nucleotide sequence.

A “truncation” shall be understood to mean that at least one nucleotide at either the 3′-end or the 5′-end of the nucleotide sequence is missing compared to the nucleic sequence of the corresponding wild type sequence or that at least one amino acid at either the N-terminus or the C-terminus of the protein is missing compared to the amino acid sequence of the corresponding wild type protein, whereby in a 3′-end or C-terminal truncation at least the first nucleotide at the 5′-end or the first amino acid at the N-terminus, respectively, is still present and in a 5′-end or N-terminal truncation at least the last nucleotide at the 3′-end or the last amino acid at the C-terminus, respectively, is still present. The 5′-end is determined by the ATG codon used as start codon in translation of a corresponding wild type nucleic acid sequence.

“Replacement” shall mean that at least one nucleotide in a nucleic acid sequence or one amino acid in a protein sequence is different compared to the corresponding wild type nucleic acid sequence or the corresponding wild type amino acid sequence, respectively, due to an exchange of a nucleotide in the coding sequence of the respective protein.

“Insertion” shall mean that the nucleic acid sequence or the amino acid sequence of a protein comprises at least one additional nucleotide or amino acid compared to the corresponding wild type nucleic acid sequence or the corresponding wild type amino acid sequence, respectively.

“Pre-mature stop codon” in context with the present invention means that a stop codon is present in a coding sequence (cds) which is closer to the start codon at the 5′-end compared to the stop codon of a corresponding wild type coding sequence.

A “mutation in a regulatory sequence”, e.g. in a promoter or enhancer of a gene, is a change of one or more nucleotides compared to the wild type sequence, e.g. by replacement, deletion or insertion of one or more nucleotides, leading for example to decreased or no mRNA transcript of the gene being made. The “promoter of a gene sequence”, accordingly is defined as a region of DNA that initiates transcription of a particular gene. Promoters are located near the genes they transcribe, on the same strand and upstream on the DNA. Promoters can be about 100-1000 base pairs long. In one aspect, the promoter is defined as the region of about 2000 base pairs or more upstream of the start codon (i.e. ATG) of the protein encoded by the gene, preferably, the promoter is the region of about 1500 base pairs upstream of the start codon, more preferably the promoter is the region of about 1000 base pairs upstream of the start codon.

As used herein, the term “operably linked” refers to a linkage of polynucleotide elements in a functional relationship. A nucleic acid is “operably linked” when it is placed into a functional relationship with another nucleic acid sequence. For instance, a promoter, or rather a transcription regulatory sequence, is operably linked to a coding sequence if it affects the transcription of the coding sequence. Operably linked means that the nucleic acid sequences being linked are typically contiguous.

“Sequence identity” and “sequence similarity” can be determined by alignment of two peptide or two nucleotide sequences using global or local alignment algorithms. Sequences may then be referred to as “substantially identical” when they are optimally aligned by for example the programs GAP or BESTFIT or the Emboss program “Needle” (using default parameters, see below) share at least a certain minimal percentage of sequence identity (as defined further below). These programs use the Needleman and Wunsch global alignment algorithm to align two sequences over their entire length, maximizing the number of matches and minimizing the number of gaps. Generally, the default parameters are used, with a gap creation penalty=10 and gap extension penalty=0.5 (both for nucleotide and protein alignments). For nucleotides the default scoring matrix used is DNAFULL and for proteins the default scoring matrix is Blosum62 (Henikoff & Henikoff, 1992, PNAS 89, 10915-10919). Sequence alignments and scores for percentage sequence identity may for example be determined using computer programs, such as EMBOSS, (as available on the Internet by ebi.ac.uk at http://www.ebi.ac.uk under /Tools/psa/emboss_needle/). Alternatively, sequence similarity or identity may be determined by searching against databases such as FASTA, BLAST, etc., but hits should be retrieved and aligned pairwise to compare sequence identity. Two proteins or two protein domains, or two nucleic acid sequences have “substantial sequence identity” if the percentage sequence identity is at least 95%, 96%, 97%, 98%, 98.3%, 98.7%, 99.0%, or 99.3% or more preferably 99.7% (as determined by Emboss “needle” using default parameters, i.e. gap creation penalty=10, gap extension penalty=0.5, using scoring matrix DNAFULL for nucleic acids and Blosum62 for proteins). Such sequences are also referred to as ‘variants’ herein, e.g. other variants of alleles causing the male sterility trait of the present invention and/or the anthocyanin absent trait of the present invention and proteins than the specific nucleic acid and amino acid sequences disclosed herein can be identified, which have the same effect on male sterility and/or the absence of anthocyanin in the hypocotyl as the plants of the present invention.

The term “hybridisation” as used herein is generally used to mean hybridisation of nucleic acids at appropriate conditions of stringency (stringent hybridisation conditions) as would be readily evident to those skilled in the art depending upon the nature of the probe sequence and target sequences. Conditions of hybridisation and washing are well-known in the art, and the adjustment of conditions depending upon the desired stringency by varying incubation time, temperature and/or ionic strength of the solution are readily accomplished. See, for example, Sambrook, J. et al., Molecular Cloning: A Laboratory Manual, 2nd edition, Cold Spring Harbor Press, Cold Spring Harbor, N.Y., 1989. The choice of conditions is dictated by the length of the sequences being hybridised, in particular, the length of the probe sequence, the relative G-C content of the nucleic acids and the amount of mismatches to be permitted. Low stringency conditions are preferred when partial hybridisation between strands that have lesser degrees of complementarity is desired. When perfect or near perfect complementarity is desired, high stringency conditions are preferred. For typical high stringency conditions, the hybridisation solution contains 6×S.S.C., 0.01 M EDTA, 1×Denhardt's solution and 0.5% SOS. hybridisation is carried out at about 68° C. for about 3 to 4 hours for fragments of cloned DNA and for about 12 to about 16 hours for total eukaryotic DNA. For lower stringencies the temperature of hybridisation is reduced to about 42° C. below the melting temperature (T_{M}) of the duplex. The T_{M} is known to be a function of the G-C content and duplex length as well as the ionic strength of the solution.

As used herein, the phrase “hybridizes” to a DNA or RNA molecule means that the molecule that hybridizes, e.g., oligonucleotide, polynucleotide, or any nucleotide sequence (in sense or antisense orientation) recognizes and hybridizes to a sequence in another nucleic acid molecule that is of approximately the same size and has enough sequence similarity thereto to effect hybridisation under appropriate conditions. For example, a 100 nucleotide long molecule from the 3′ coding or non-coding region of a gene will recognize and hybridize to an approximately 100 nucleotide portion of a nucleotide sequence within the 3′ coding or non-coding region of that gene or any other plant gene so long as there is about 70% or more sequence similarity between the two sequences. It is to be understood that the size of the corresponding portion will allow for some mismatches in hybridisation such that the corresponding portion may be smaller or larger than the molecule which hybridizes to it, for example 20-30% larger or smaller, preferably no more than about 12-15% larger or smaller.

As used herein, the phrase “a sequence comprising at least 95% sequence identity” or “a sequence comprising at least 95% amino acid sequence identity” or “a sequence comprising at least 95% nucleotide sequence identity” means a sequence having at least 95% e.g. at least 96%, 97%, 98%, 98.3%, 98.7%, 99.0%, or 99.3% or more preferably 99.7% sequence identity when compared with the reference sequence that is indicated. Sequence identity can be determined according the methods described herein.

A “fragment” of the gene or DNA sequence refers to any subset of the molecule, e.g., a shorter polynucleotide or oligonucleotide. In one aspect the fragment comprises the mutation as defined by the invention.

A “variant” of the gene or DNA refers to a molecule substantially similar to either the entire gene or a fragment thereof, such as a nucleotide substitution variant having one or more substituted nucleotides, but which maintains the ability to hybridize with the particular gene or to encode mRNA transcript which hybridizes with the native DNA. Preferably the variant comprises the mutant allele as defined by the invention.

As used herein, the term “plant” includes the whole plant or any parts or derivatives thereof, such as plant organs (e.g., harvested or non-harvested flowers, leaves, etc.), plant cells, plant protoplasts, plant cell or tissue cultures from which whole plants can be regenerated, regenerable or non-regenerable plant cells, plant calli, plant cell clumps, and plant cells that are intact in plants, or parts of plants, such as embryos, pollen, ovules, ovaries (e.g., harvested tissues or organs), flowers, leaves, seeds, tubers, clonally propagated plants, roots, stems, cotyledons, hypocotyls, root tips and the like. Also, any developmental stage is included, such as seedlings, immature and mature, etc. Preferably, the plant part or derivative comprises the MS10 gene or locus and/or the AA gene or locus as defined by the current invention.

A “plant line” or “breeding line” refers to a plant and its progeny.

“Plant variety” is a group of plants within the same botanical taxon of the lowest grade known, which (irrespective of whether the conditions for the recognition of plant breeders rights are fulfilled or not) can be defined on the basis of the expression of characteristics that result from a certain genotype or a combination of genotypes, can be distinguished from any other group of plants by the expression of at least one of those characteristics, and can be regarded as an entity, because it can be multiplied without any change. Therefore, the term “plant variety” cannot be used to denote a group of plants, even if they are of the same kind, if they are all characterized by the presence of 1 locus or gene (or a series of phenotypical characteristics due to this single locus or gene), but which can otherwise differ from one another enormously as regards the other loci or genes. “F1, F2, etc.” refers to the consecutive related generations following a cross between two parent plants or parent lines. The plants grown from the seeds produced by crossing two plants or lines is called the F1 generation. Selfing the F1 plants results in the F2 generation, etc. “F1 hybrid” plant (or F1 seed, or hybrid) is the generation obtained from crossing two inbred parent lines. “Selfing”, accordingly, refers to the self-pollination of a plant, i.e. to the union of gametes from the same plant.

“Backcrossing” refers to a breeding method by which a (single) trait, such as the male sterility trait and/or the anthocyanin absent trait, can be transferred from one genetic background (also referred to as “donor” generally, but not necessarily, this is an inferior genetic background) into another genetic background (also referred to as “recurrent parent”; generally, but not necessarily, this is a superior genetic background). An offspring of a cross (e.g. an F1 plant obtained by crossing a first plant of a certain plant species comprising the mutant allele of the present invention with a second plant of the same plant species or of a different plant species that can be crossed with said first plant species wherein said second plant species does not comprise the mutant allele of the present invention; or an F2 plant or F3 plant, etc., obtained by selfing the F1) is “backcrossed” to a parent plant of said second plant species. After repeated backcrossing, the trait of the donor genetic background, e.g. the mutant allele conferring the male sterility trait and/or the anthocyanin absent trait as described herein, will have been incorporated into the recurrent genetic background. The terms “gene converted” or “conversion plant” or “single locus conversion” in this context refer to plants which are developed by backcrossing wherein essentially all of the desired morphological and/or physiological characteristics of the recurrent parent are recovered in addition to the one or more genes transferred from the donor parent. The plants grown from the seeds produced by backcrossing of the F1 plants with the second parent plant line is referred to as the “BC1 generation”. Plants from the BC1 population may be selfed resulting in the BC1F2 generation or backcrossed again with the cultivated parent plant line to provide the BC2 generation. An “M1 population” is a plurality of mutagenized seeds/plants of a certain plant line. “M2, M3, M4, etc.” refers to the consecutive generations obtained following selfing of a first mutagenized seed/plant (M1).

Solanum lycopersicum plants, also referred herein to as “plants of the species Solanum lycopersicum” or “tomato plants”, are perennial in their native habitat but cultivated as an annual. Cultivated Solanum lycopersicum plants typically grow to 1-3 meters (3-10 ft) in height. Tomato fruits are botanically berry-type fruits, they are considered culinary vegetables. Fruit size varies according to cultivar, with a width range of about 1-10 cm (about 0.5-4 inches). Solanum lycopersicum is also known as Lycopersicon lycopersicum (L.) H. Karst. or Lycopersicon esculentum Mill. The term “cultivated tomato plant” or “cultivated tomato” refers to plants of Solanum lycopersicum, e.g. varieties, breeding lines or cultivars of the species S. lycopersicum, cultivated by humans and having good agronomic characteristics. The term “wild relatives of Solanum lycopersicum” or “wild relatives of tomato” include S. arcanum, S. chmielewskii, S. neorickii (=L. parviflorum), S. cheesmaniae, S. galapagense, S. pimpinellifolium, S. chilense, S. corneliomulleri, S. habrochaites (=L. hirsutum), S. huaylasense, S. sisymbriifolium, S. peruvianum, S. hirsutum or S. pennellii. Tomato and the wild relatives of tomato is/are diploid and has/have 12 pairs of homologous chromosomes, numbered 1 to 12.

The term “cultivated plant” or “cultivar” refers to plants of a given species, e.g. varieties, breeding lines or cultivars of the said species, cultivated by humans and having good agronomic characteristics. The so-called heirloom varieties or cultivars, i.e. open pollinated varieties or cultivars commonly grown during earlier periods in human history and often adapted to specific geographic regions, are in one aspect of the invention encompassed herein as cultivated plants. The term “cultivated plant” does not encompass wild plants. “Wild plants” include for example wild accessions.

The term “food” is any substance consumed to provide nutritional support for the body. It is usually of plant or animal origin, and contains essential nutrients, such as carbohydrates, fats, proteins, vitamins, or minerals. The substance is ingested by an organism and assimilated by the organism's cells in an effort to produce energy, maintain life, or stimulate growth. The term food includes substance consumed to provide nutritional support for both the human and animal body.

“Vegetative propagation” or “clonal propagation” refers to propagation of plants from vegetative tissue, e.g. by propagating plants from cuttings or by in vitro propagation. In vitro propagation involves in vitro cell or tissue culture and regeneration of a whole plant from the in vitro culture. Clones (i.e. genetically identical vegetative propagations) of the original plant can thus be generated by in vitro culture. “Cell culture” or “tissue culture” refers to the in vitro culture of cells or tissues of a plant. “Regeneration” refers to the development of a plant from cell culture or tissue culture or vegetative propagation. “Non-propagating cell” refers to a cell which cannot be regenerated into a whole plant.

The term “meiotic recombination” refers to the genetic recombination involving the pairing of homologous chromosomes that occurs in eukaryotes during meiosis. The pairing of homologous chromosomes may be followed by information transfer between said chromosomes. This information transfer may occur without physical exchange (a section of genetic material is copied from one chromosome to another, without the donating chromosome being changed) or by the breaking and re-joining of DNA strands, which forms a newly recombined DNA molecule.

“Average” refers herein to the arithmetic mean.

It is understood that comparisons between different plant lines involves growing a number of plants of a line (or variety) (e.g. at least 5 plants, preferably at least 10 plants per line) under the same conditions as the plants of one or more control plant lines (preferably wild type plants) and the determination of differences, preferably statistically significant differences, between the plant lines when grown under the same environmental conditions. Preferably the plants are of the same line or variety.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”. It is further understood that, when referring to “sequences” herein, generally the actual physical molecules with a certain sequence of subunits (e.g. amino acids or nucleic acids) are referred to.

### Plants of the Invention

The present invention provides a plant of the species Solanum lycopersicum comprising in its genome at least one chromosome comprising a mutant allele of the wild type male sterility 10 (MS10) gene and a mutant allele of the wild type anthocyanin absent (AA) gene wherein in said plant the meiotic recombination frequency is reduced between said mutant allele of the wild type MS10 gene and said mutant allele of the wild type AA gene when compared to the meiotic recombination frequency between the MS10 gene and the AA gene in a wild type Solanum lycopersicum plant, wherein the wild type MS10 gene encodes a protein comprising at least 95% amino acid sequence identity to SEQ ID NO: 1 and the mutant allele of the wild type MS10 gene results in no expression or reduced expression of the wild type gene and/or the mutant allele of the wild type MS10 gene encodes a protein having loss-of-function or reduced function when compared to the wild type protein, and wherein the wild type AA gene encodes a protein comprising at least 95% amino acid sequence identity to SEQ ID NO: 3 and the mutant allele of the wild type AA gene results in no expression or reduced expression of the wild type gene and/or the mutant allele of the wild type AA gene encodes a protein having loss-of-function or reduced function when compared to the wild type protein.

The male sterile plants according to the present invention thus can be reliably identified and/or selected based on the phenotype of the hypocotyls of said plant, i.e. by determining whether anthocyanin coloring is absent in the hypocotyls, without having to confirm the genotype of the MS10 allele to prevent the selection of plants that show the anthocyanin absent trait but not the male sterility trait due to meiotic recombination. The currently available Solanum lycopersicum plants wherein a male sterility trait is combined with a further trait for visual identification of the male sterile plants do not allow a fully reliable selection of the male sterile plants due to the too frequent occurrence of meiotic recombination events leading to offspring wherein the linkage between the male sterility trait and the trait for visual identification is lost.

The present invention accordingly provides a tomato plant wherein meiotic recombination is suppressed between the mutant allele of the wild type MS10 gene and the mutant allele of the wild type AA gene. This accordingly means that in a heterozygous plant according to the present invention (i.e. a plant comprising one mutant chromosome 2 comprising the mutant MS10 allele and the mutant AA allele comprising an inversion and/or a deletion in the genomic region between said mutant MS10 allele and said mutant AA allele and one wild type chromosome 2 comprising a wild type MS10 allele and a wild type AA allele) the meiotic recombination frequency between the mutant MS10 allele and the mutant AA allele is reduced when compared to the meiotic recombination frequency between the MS10 gene and the AA gene in a wild type Solanum lycopersicum plant. The meiotic recombination frequency between the MS10 gene and the AA gene in a wild type Solanum lycopersicum plant is identical to the meiotic recombination frequency between the mutant MS10 allele and the mutant AA allele in a heterozygous ms10-aa plant according to the prior art, e.g. as described in Zhang et al. Mol Breeding (2016) 36:107 which does not comprise an inversion and/or a deletion in the genomic region between the mutant MS10 allele and the mutant AA allele. The term “suppression of meiotic recombination” or “reduction of the meiotic recombination frequency” in the context of the present invention indicates that the observed rate of meiotic recombination in a heterozygous plant according to the present invention is less than the rate of meiotic recombination that is expected based on the physical distance between the locus of the MS10 gene and the locus of the AA gene in a wild type plant. The plant according to the present invention accordingly preferably is a plant of the species Solanum lycopersicum comprising in its genome at least one chromosome comprising a mutant allele of the wild type male sterility 10 gene (mutant MS10 allele) and a mutant allele of the wild type anthocyanin absent gene (mutant AA allele) wherein said plant comprises an inversion and/or a deletion in the genomic region between said mutant MS10 allele and said mutant AA allele. A plant comprising an inversion is preferred over a plant comprising a deletion since said deletion may lead to additional undesired characteristics in the event said deletion leads to the loss of protein function of other genes than the MS10 gene and the AA gene.

In a plant according to the invention the observed rate of meiotic recombination in a heterozygous plant is less than the rate of meiotic recombination that is expected based on the physical distance between the locus of the MS10 gene and the locus of the AA gene in a wild type plant. In other words, the meiotic recombination frequency in the plants and the methods according to the present invention is considered to be reduced when compared to a Solanum lycopersicum plant having a wild type chromosome 2 when the genetic distance between the locus of the mutant allele of the wild type MS10 gene and the locus of the mutant allele of the wild type AA gene is less than the genetic distance between the locus of wild type allele of the wild type MS10 gene and the locus of the wild type allele of the wild type AA gene. The genetic distance between the locus of wild type allele of the wild type MS10 gene and the locus of the wild type allele of the wild type AA gene is about 7 cM. In the present invention, accordingly, a reduced meiotic recombination frequency corresponds to a genetic distance between the mutant ms10 allele and the mutant aa allele which is less than 7 cM, e.g. no more than 6.5 cM, no more than 6 cM, no more than 5.5 cM, no more than 5 cM, no more than 4.5 cM, no more than 4 cM, no more than 3.5 no more than cM, no more than 3 cM, no more than 2.5 cM, no more than 2 cM, no more than 1.5 cM, no more than 1 cM, no more than 0.9 cM, no more than 0.8 cM no more than 0.7 cM, no more than 0.6 cM, no more than 0.5 cM, no more than 0.4 cM, no more than 0.3 cM, no more than 0.2 cM, or no more than 0.1 cM. Preferably, the reduced meiotic recombination frequency corresponds to a genetic distance between the mutant ms10 allele and the mutant aa allele of less than 6 cM. More preferably, the reduced meiotic recombination frequency corresponds to a genetic distance between the mutant ms10 allele and the mutant aa allele of no more than 1 cM. Most preferably, the reduced meiotic recombination frequency corresponds to a genetic distance between the mutant ms10 allele and the mutant aa allele of no more than 0.1 cM.

There are several ways to provide a plant wherein the meiotic recombination frequency is reduced between two loci. In the context of the present invention, meiotic recombination between two loci, e.g. between the MS10 gene and the AA gene, is considered to be suppressed when the frequency of meiotic recombination between said loci is reduced when compared with a wild type plant. In one non-limiting example, a deletion may be induced in the genome resulting in a reduction of the physical distance between the locus of the mutant allele of the wild type MS10 gene and the locus of the mutant allele of the wild type AA gene. Alternatively, meiotic recombination between two loci can be suppressed by the introgression of an introgression fragment located between the two loci, wherein said introgression fragment has a sufficiently reduced homology compared to the wild type fragment to result in a reduction of the meiotic recombination. In a particularly preferred alternative, meiotic recombination between two loci can be suppressed as the result of an inversion between said two loci in one of the chromosome pairs. Due to the inversion, there is no homology between the two loci, which is required for meiotic recombination to occur. The present invention accordingly preferably provides a plant of the species Solanum lycopersicum comprising in its genome at least one chromosome comprising a mutant allele of the wild type MS10 gene and a mutant allele of the wild type AA gene wherein the genomic DNA region between the MS10 gene and the AA gene comprises an inversion resulting in that the meiotic recombination frequency between the MS10 gene and the AA gene is reduced when compared to the meiotic recombination frequency between the MS10 gene and the AA gene in a wild type Solanum lycopersicum plant, wherein the wild type MS10 gene encodes a protein comprising at least 95% amino acid sequence identity to SEQ ID NO: 1 and the mutant allele of the wild type MS10 gene results in no expression or reduced expression of the wild type gene and/or the mutant allele of the wild type MS10 gene encodes a protein having loss-of-function or reduced function when compared to the wild type protein, and wherein the wild type AA gene encodes a protein comprising at least 95% amino acid sequence identity to SEQ ID NO: 3 and the mutant allele of the wild type AA gene results in no expression or reduced expression of the wild type gene and/or the mutant allele of the wild type AA gene encodes a protein having loss-of-function or reduced function when compared to the wild type protein.

Accordingly, the present invention provides a plant of the species Solanum lycopersicum comprising in its genome at least one chromosome comprising a mutant allele of the wild type male sterility 10 (MS10) gene and a mutant allele of the wild type anthocyanin absent (AA) gene wherein in said plant the meiotic recombination frequency is reduced between said mutant allele of the wild type MS10 gene and said mutant allele of the wild type AA gene when compared to the meiotic recombination frequency between the MS10 gene and the AA gene in a wild type Solanum lycopersicum plant, wherein the wild type MS10 gene encodes a protein comprising at least 95% amino acid sequence identity to SEQ ID NO: 1, e.g. 96%, 97%, 98%, 98.3%, 98.7%, 99.0%, or 99.3% or more preferably 99.7% sequence identity to SEQ ID NO: 1, (as determined using methods discloses elsewhere herein) and the mutant allele of the wild type MS10 gene results in no expression or reduced expression of the wild type gene and/or the mutant allele of the wild type MS10 gene encodes a protein having loss-of-function or reduced function when compared to the wild type protein, and wherein the wild type AA gene encodes a protein comprising at least 95% amino acid sequence identity to SEQ ID NO: 3, e.g. 96%, 97%, 98%, 98.3%, 98.7%, 99.0%, or 99.3% or more preferably 99.7% sequence identity to SEQ ID NO: 3, (as determined using methods discloses elsewhere herein) and the mutant allele of the wild type AA gene results in no expression or reduced expression of the wild type gene and/or the mutant allele of the wild type AA gene encodes a protein having loss-of-function or reduced function when compared to the wild type protein.

The Solanum lycopersicum plant of the present invention comprising in its genome at least one copy of the chromosome comprising a mutant allele of the wild type MS10 gene and a mutant allele of the wild type AA gene, wherein the meiotic recombination frequency is reduced between said mutant allele of the wild type MS10 gene and said mutant allele of the wild type AA gene when compared to the meiotic recombination frequency between the MS10 gene and the AA gene in a wild type Solanum lycopersicum plant. Accordingly, the plant of the present invention may heterozygous for the male sterility trait and anthocyanin absent trait of the present invention and thus comprise one wild type chromosome comprising a wild type allele of the wild type MS10 gene and a wild type allele of the wild type AA gene in addition to the chromosome comprising a mutant allele of the wild type MS10 gene and a mutant allele of the wild type AA gene. The heterozygous plants accordingly are characterized by the chromosome comprising the mutant ms10 allele and the mutant aa allele between which the meiotic recombination frequency is reduced when compared to the meiotic recombination frequency between the MS10 gene and the AA gene in a wild type Solanum lycopersicum plant. Homozygous plants showing the male sterility phenotype and the anthocyanin absent phenotype can be readily obtained from heterozygous plants e.g. by selfing. The plant of the present invention preferably is homozygous for the mutant allele of the wild type MS10 gene and homozygous for the mutant allele of the wild type AA gene.

The mutant allele of the wild type MS10 gene according to the present invention results in no expression or reduced expression of the wild type gene and/or the mutant allele of the wild type MS10 gene encodes a protein having loss-of-function or reduced function when compared to the wild type protein. Preferably, the mutant allele of the wild type MS10 gene according to the present invention results in no expression of the wild type gene and/or the mutant allele of the wild type MS10 gene encodes a protein having loss-of-function when compared to the wild type protein. Said mutant allele of the wild type MS10 resulting in no expression and/or the mutant allele of the wild type MS10 gene encodes a protein having loss-of-function leads to complete loss of protein function and thus inevitably induces male sterility when present in homozygous form. Alternatively, the mutant allele of the wild type MS10 gene according to the present invention results in reduced expression of the wild type gene and/or the mutant allele of the wild type MS10 gene encodes a protein having reduced function when compared to the wild type protein. Said mutant allele of the wild type MS10 resulting in reduced expression and/or the mutant allele of the wild type MS10 gene encodes a protein having reduced function leads to a sufficient reduction in protein function to induce male sterility when present in homozygous form. Accordingly, the mutant allele of the wild type male MS10 gene (mutant ms10 allele) according to the present invention preferably induces male sterility when present in homozygous form.

The mutant allele of the wild type AA gene according to the present invention results in no expression or reduced expression of the wild type gene and/or the mutant allele of the wild type AA gene encodes a protein having loss-of-function or reduced function when compared to the wild type protein. Preferably, the mutant allele of the wild type AA gene according to the present invention results in no expression of the wild type gene and/or the mutant allele of the wild type AA gene encodes a protein having loss-of-function when compared to the wild type protein. Said mutant allele of the wild type AA gene resulting in no expression and/or the mutant allele of the wild type AA gene encodes a protein having loss-of-function leads to complete loss of protein function and thus inevitably induces anthocyanin absent hypocotyls when present in homozygous form. Alternatively, the mutant allele of the wild type AA gene according to the present invention results in reduced expression of the wild type gene and/or the mutant allele of the wild type AA gene encodes a protein having reduced function when compared to the wild type protein. Said mutant allele of the wild type AA gene resulting in reduced expression and/or the mutant allele of the wild type AA gene encodes a protein having reduced function leads to a sufficient reduction in protein function to induce anthocyanin absent hypocotyls when present in homozygous form. Accordingly, the mutant allele of the wild type AA gene (mutant aa allele) according to the present invention preferably induces the absence of anthocyanin in the hypocotyls when present in homozygous form.

The Solanum lycopersicum plant of the present invention preferably is homozygous for the mutant allele of the wild type MS10 gene and a mutant allele of the wild type AA gene, wherein the meiotic recombination frequency is reduced between said mutant allele of the wild type MS10 gene and said mutant allele of the wild type AA gene when compared to the meiotic recombination frequency between the MS10 gene and the AA gene in a wild type Solanum lycopersicum plant. Accordingly, the Solanum lycopersicum plant of the present invention preferably is homozygous for the chromosome comprising a mutant allele of the wild type MS10 gene and a mutant allele of the wild type AA gene, wherein the meiotic recombination frequency is reduced between said mutant allele of the wild type MS10 gene and said mutant allele of the wild type AA gene when compared to the meiotic recombination frequency between the MS10 gene and the AA gene in a wild type Solanum lycopersicum plant. Preferably. the plant according to the present invention is an inbred plant, a dihaploid plant or a hybrid plant. In one aspect, accordingly, the present invention provides that the plant of the present invention is an inbred plant. Such an inbred plant is highly homozygous, for instance by repeated selfing crossing steps. Such an inbred plant may be very useful as a parental plant for the production of F1 hybrid seed. In one aspect, the disclosure provides for haploid plants and/or dihaploid (double haploid) plants of plant of the invention are encompassed herein, which comprise the mutant ms10 allele and the mutant aa allele as described herein. Haploid and dihaploid plants can for example be produced by anther or microspore culture and regeneration into a whole plant. For dihaploid production chromosome doubling may be induced using known methods, such as colchicine treatment or the like. So, in one aspect a Solanum lycopersicum plant is provided, comprising the male sterility and anthocyanin absent phenotype as described, wherein the plant is a dihaploid plant. The present invention further provides hybrid plants, which may have advantages such as improved uniformity, vitality and/or disease tolerance.

The plants provided by the present invention may be used to produce fruits, particularly for F1 hybrid seed production. The present invention thus provides the use of a plant of the species Solanum lycopersicum as provided herein for seed production. Particularly the fruits produced by the plants of the present invention can be advantageously used for seed production since the seed production does not require manual emasculation of the flowers to prevent self-pollination.

The plants provided by the present invention may be used to produce propagation material. Such propagation material comprises propagation material suitable for and/or resulting from sexual reproduction, such as pollen and seeds. Such propagation material comprises propagation material suitable for and/or resulting from asexual or vegetative reproduction including, but not limited to cuttings, grafts, tubers, cell culture and tissue culture. The present invention thus further provides the use of a plant of the species Solanum lycopersicum as provided herein as a source of propagation material.

The present invention provides seed from which any plant according to the invention can be grown. Furthermore, the invention provides a plurality of such seed. A seed of the invention can be distinguished from other seeds due to the presence of at least one chromosome comprising a mutant allele of the wild type male sterility 10 (MS10) gene and a mutant allele of the wild type anthocyanin absent (AA) gene wherein in said plant the meiotic recombination frequency is reduced between said mutant allele of the wild type MS10 gene and said mutant allele of the wild type AA gene when compared to the meiotic recombination frequency between the MS10 gene and the AA gene in a wild type Solanum lycopersicum plant as described herein, either phenotypically (based on plants having the male sterility trait in combination with the anthocyanin absent trait of the present invention) and/or using molecular methods to detect the mutant allele in the cells or tissues, such as molecular genotyping methods to detect the mutant allele of the present invention or sequencing. Seeds include for example seeds produced by a plant of the invention which is heterozygous for the mutant allele after self-pollination and optionally selection of those seeds which comprise one or two copies of the mutant ms10 and aa alleles (e.g. by non-destructive seed sampling methods and analysis of the presence of the mutant ms10 and aa alleles), or seed produced after cross-pollination, e.g. pollination of a plant of the invention with pollen from another solanaceous plant, preferably from another Solanum lycopersicum plant, or pollination of another Solanum lycopersicum plant with pollen of a plant of the invention.

Particularly, the present invention provides pollen or seed produced by the plant according to the present invention, or seed from which a plant of the invention can be grown, wherein said plant is a plant of the species Solanum lycopersicum comprising in its genome at least one chromosome comprising a mutant allele of the wild type male sterility 10 (MS10) gene and a mutant allele of the wild type anthocyanin absent (AA) gene wherein in said plant the meiotic recombination frequency is reduced between said mutant allele of the wild type MS10 gene and said mutant allele of the wild type AA gene when compared to the meiotic recombination frequency between the MS10 gene and the AA gene in a wild type Solanum lycopersicum plant, wherein the wild type MS10 gene encodes a protein comprising at least 95% amino acid sequence identity to SEQ ID NO: 1, e.g. 96%, 97%, 98%, 98.3%, 98.7%, 99.0%, or 99.3% or more preferably 99.7% sequence identity to SEQ ID NO: 1, (as determined using methods discloses elsewhere herein) and the mutant allele of the wild type MS10 gene results in no expression or reduced expression of the wild type gene and/or the mutant allele of the wild type MS10 gene encodes a protein having loss-of-function or reduced function when compared to the wild type protein, and wherein the wild type AA gene encodes a protein comprising at least 95% amino acid sequence identity to SEQ ID NO: 3, e.g. 96%, 97%, 98%, 98.3%, 98.7%, 99.0%, or 99.3% or more preferably 99.7% sequence identity to SEQ ID NO: 3, (as determined using methods discloses elsewhere herein) and the mutant allele of the wild type AA gene results in no expression or reduced expression of the wild type gene and/or the mutant allele of the wild type AA gene encodes a protein having loss-of-function or reduced function when compared to the wild type protein.

Particularly, the present invention provides pollen or seed produced by the plant according to the present invention, or seed from which a plant of the invention can be grown, wherein the pollen or seed comprises the mutant allele of the wild type MS10 gene and a mutant allele of the wild type AA gene, wherein the meiotic recombination frequency is reduced between said mutant allele of the wild type MS10 gene and said mutant allele of the wild type AA gene when compared to the meiotic recombination frequency between the MS10 gene and the AA gene in a wild type Solanum lycopersicum plant. The present invention accordingly provides seed from which a plant according to the present invention can be grown.

In one aspect, a plurality of seed is packaged into a container (e.g. a bag, a carton, a can etc.). Containers may be any size. The seeds may be pelleted prior to packing (to form pills or pellets) and/or treated with various compounds, including seed coatings.

In a further aspect a plant part, obtained from (obtainable from) a plant of the invention is provided herein, and a container or a package comprising said plant part.

Particularly, the present invention provides a part from the plant of the present invention, wherein the part comprises in its genome at least one chromosome comprising a mutant allele of the wild type MS10 gene and a mutant allele of the wild type AA gene wherein in said plant the meiotic recombination frequency is reduced between said mutant allele of the wild type MS10 gene and said mutant allele of the wild type AA gene when compared to the meiotic recombination frequency between the MS10 gene and the AA gene in a wild type Solanum lycopersicum plant, wherein the wild type MS10 gene encodes a protein comprising at least 95% amino acid sequence identity to SEQ ID NO: 1, e.g. 96%, 97%, 98%, 98.3%, 98.7%, 99.0%, or 99.3% or more preferably 99.7% sequence identity to SEQ ID NO: 1, (as determined using methods discloses elsewhere herein) and the mutant allele of the wild type MS10 gene results in no expression or reduced expression of the wild type gene and/or the mutant allele of the wild type MS10 gene encodes a protein having loss-of-function or reduced function when compared to the wild type protein, and wherein the wild type AA gene encodes a protein comprising at least 95% amino acid sequence identity to SEQ ID NO: 3, e.g. 96%, 97%, 98%, 98.3%, 98.7%, 99.0%, or 99.3% or more preferably 99.7% sequence identity to SEQ ID NO: 3, (as determined using methods discloses elsewhere herein) and the mutant allele of the wild type AA gene results in no expression or reduced expression of the wild type gene and/or the mutant allele of the wild type AA gene encodes a protein having loss-of-function or reduced function when compared to the wild type protein. Preferably, the plant part is selected from the group consisting of a leaf, anther, pistil, stem, petiole, root, ovule, pollen, microspore, protoplast, callus, tissue, seed, flower, cotyledon, hypocotyl, embryo and cell. The various stages of development of aforementioned plant parts are comprised in the invention. The present invention accordingly provides a part of the plant according present invention. Preferably such a plant part according to the present invention is a leaf, anther, pistil, stem, petiole, root, ovule, pollen, microspore, protoplast, callus, tissue, seed, flower, cotyledon, hypocotyl, embryo or cell.

In a further aspect, the plant part is a plant cell. In still a further aspect, the plant part is a non-regenerable cell or a regenerable cell. In another aspect the plant cell is a somatic cell.

A non-regenerable cell is a cell which cannot be regenerated into a whole plant through in vitro culture. The non-regenerable cell may be in a plant or plant part (e.g. leaves) of the invention. The non-regenerable cell may be a cell in a seed, or in the seedcoat of said seed. Mature plant organs, including a mature leaf, a mature stem or a mature root, contain at least one non-regenerable cell.

In a further aspect the plant cell is a reproductive cell, such as an ovule or a cell which is part of a pollen. In an aspect, the pollen cell is the vegetative (non-reproductive) cell, or the sperm cell (Tiezzi, Electron Microsc. Review, 1991). Such a reproductive cell is haploid. When it is regenerated into whole a plant, it comprises the haploid genome of the starting plant. If chromosome doubling occurs (e.g. through chemical treatment), a double haploid plant can be regenerated. In one aspect the plant of the invention comprising the mutant allele of the wild type MS10 gene and a mutant allele of the wild type AA gene is a haploid or a double haploid Solanum lycopersicum plant according to the present invention.

Moreover, there is provided an in vitro cell culture or tissue culture of the Solanum lycopersicum plant of the invention in which the cell- or tissue culture is derived from a plant part described above, such as, for example and without limitation, a leaf, a pollen, an embryo, cotyledon, hypocotyls, callus, a root, a root tip, an anther, a flower, a seed or a stem, or a part of any of them, or a meristematic cell, a somatic cell, or a reproductive cell.

The present invention further provides a vegetatively propagated plant, wherein said plant is propagated from a plant part according to the present invention.

Further, isolated cells, in vitro cell cultures and tissue cultures, protoplast cultures, plant parts, harvested material (e.g. harvested tomato fruits), pollen, ovaries, flowers, seeds, stamen, flower parts, etc. comprising in each cell at least one chromosome comprising the mutant ms10 allele and the mutant aa allele of the present invention are provided. Thus, when said cells or tissues are regenerated or grown into a whole Solanum lycopersicum plant, the plant comprises the mutant allele capable of inducing male sterility and anthocyanin absent hypocotyls when present in homozygous form.

Thus, also an in vitro cell culture and/or tissue culture of cells or tissues of plants of the invention is provided. The cell or tissue culture can be treated with shooting and/or rooting media to regenerate a Solanum lycopersicum plant.

Also, vegetative or clonal propagation of plants according to the invention is encompassed herein. Many different vegetative propagation techniques exist. Cuttings (nodes, shoot tips, stems, etc.) can for example be used for in vitro culture as described above. Also, other vegetative propagation techniques exist and can be used, such as grafting, or air layering. In air layering a piece of stem is allowed to develop roots while it is still attached to the parent plant and once enough roots have developed the clonal plant is separated from the parent.

Thus, in one aspect a method is provided comprising:
(a) obtaining a part of a plant of the invention (e.g. cells or tissues, e.g. cuttings),
(b) vegetatively propagating said plant part to generate an identical plant from the plant part.

Thus, also the use of vegetative plant parts of plants of the invention for clonal/vegetative propagation is an embodiment of the invention. In one aspect a method is provided for vegetatively reproducing a Solanum lycopersicum plant of the invention comprising two copies of the chromosome comprising the mutant ms10 allele and the mutant aa allele of the present invention is provided, wherein the meiotic recombination frequency is reduced between said mutant ms10 allele and said mutant aa allele when compared to the meiotic recombination frequency between the MS10 gene and the AA gene in a wild type Solanum lycopersicum plant as described herein. Also a vegetatively produced plant comprising two copies of the chromosome comprising the mutant ms10 allele and the mutant aa allele of the present invention are provided is provided, wherein the meiotic recombination frequency is reduced between said mutant ms10 allele and said mutant aa allele when compared to the meiotic recombination frequency between the MS10 gene and the AA gene in a wild type Solanum lycopersicum plant as described herein.

In another aspect a plant of the invention, comprising two copies of the chromosome comprising the mutant ms10 allele and the mutant aa allele according to the invention and wherein the meiotic recombination frequency is reduced between said mutant ms10 allele and said mutant aa allele when compared to the meiotic recombination frequency between the MS10 gene and the AA gene in a wild type Solanum lycopersicum plant as described herein, is propagated by somatic embryogenesis techniques.

Also provided is a Solanum lycopersicum plant regenerated from any of the above-described plant parts, or regenerated from the above-described cell or tissue cultures, said regenerated plant comprising in its genome at least one chromosome comprising a mutant allele of the wild type MS10 gene and a mutant allele of the wild type AA gene wherein in said plant the meiotic recombination frequency is reduced between said mutant allele of the wild type MS10 gene and said mutant allele of the wild type AA gene, wherein the wild type MS10 gene encodes a protein comprising at least 95% amino acid sequence identity to SEQ ID NO: 1, e.g. 96%, 97%, 98%, 98.3%, 98.7%, 99.0%, or 99.3% or more preferably 99.7% sequence identity to SEQ ID NO: 1, (as determined using methods discloses elsewhere herein) and the mutant allele of the wild type MS10 gene results in no expression or reduced expression of the wild type gene and/or the mutant allele of the wild type MS10 gene encodes a protein having loss-of-function or reduced function when compared to the wild type protein, and wherein the wild type AA gene encodes a protein comprising at least 95% amino acid sequence identity to SEQ ID NO: 3, e.g. 96%, 97%, 98%, 98.3%, 98.7%, 99.0%, or 99.3% or more preferably 99.7% sequence identity to SEQ ID NO: 3, (as determined using methods discloses elsewhere herein) and the mutant allele of the wild type AA gene results in no expression or reduced expression of the wild type gene and/or the mutant allele of the wild type AA gene encodes a protein having loss-of-function or reduced function when compared to the wild type protein. Preferably, the regenerated plant is homozygous for the chromosome comprising the mutant ms10 allele and the mutant aa allele of the present invention, wherein the meiotic recombination frequency is reduced between said mutant ms10 allele and said mutant aa allele when compared to the meiotic recombination frequency between the MS10 gene and the AA gene in a wild type Solanum lycopersicum plant as described herein and thus is male sterile in combination with having anthocyanin absent hypocotyls.

Methods of Identifying and/or Selecting a Plant or Plant Part

In another embodiment, plants and parts of Solanum lycopersicum plants of the invention, and progeny of Solanum lycopersicum plant of the invention are provided, e.g., grown from seeds, produced by sexual or vegetative reproduction, regenerated from the above-described plant parts, or regenerated from cell or tissue culture, in which the reproduced (seed propagated or vegetatively propagated) plant comprises at least one chromosome comprising the mutant ms10 allele and the mutant aa allele of the present invention, wherein the meiotic recombination frequency is reduced between said mutant ms10 allele and said mutant aa allele when compared to the meiotic recombination frequency between the MS10 gene and the AA gene in a wild type Solanum lycopersicum plant as described herein.

The present invention further provides a plant of the species Solanum lycopersicum grown from the seed as described herein. The present invention thus provides a Solanum lycopersicum plant grown from seeds obtained from the method for producing a Solanum lycopersicum plant as described herein.

Furthermore, the invention provides progeny comprising or retaining the male sterility trait and the anthocyanin absent trait as described herein (conferred by the mutant ms10 allele and the aa allele, respectively), such as progeny obtained by, e.g., selfing one or more times and/or cross-pollinating a plant of the invention with another Solanum lycopersicum plant of a different variety or breeding line of the same plant species (or of a plant species that can be crossed with the Solanum lycopersicum plant of the present invention), or with a Solanum lycopersicum plant of the invention one or more times. In particular, the invention provides progeny homozygous for the chromosome comprising the mutant ms10 allele and the mutant aa allele of the present invention, wherein the meiotic recombination frequency is reduced between said mutant ms10 allele and said mutant aa allele when compared to the meiotic recombination frequency between the MS10 gene and the AA gene in a wild type Solanum lycopersicum plant as described herein and thus is male sterile in combination with having anthocyanin absent hypocotyls. In one aspect the invention relates to for a progeny plant comprising the chromosome comprising the mutant ms10 allele and the mutant aa allele of the present invention, wherein the meiotic recombination frequency is reduced between said mutant ms10 allele and said mutant aa allele when compared to the meiotic recombination frequency between the MS10 gene and the AA gene in a wild type Solanum lycopersicum plant as described herein, such as a progeny plant that is produced from a Solanum lycopersicum plant comprising the chromosome comprising the mutant ms10 allele and the mutant aa allele, wherein the meiotic recombination frequency is reduced between said mutant ms10 allele and said mutant aa allele when compared to the meiotic recombination frequency between the MS10 gene and the AA gene in a wild type Solanum lycopersicum plant by one or more methods selected from the group consisting of: selfing, crossing, mutation, double haploid production or transformation. Mutations preferable are human induced mutations or somaclonal mutations. In one embodiment, plants or seeds of the invention may also be mutated (by e.g. irradiation, chemical mutagenesis, heat treatment, TILLING, targeted mutagenesis, etc.) and/or mutated seeds or plants may be selected (e.g. somaclonal variants, etc.) in order to change one or more characteristics of the plants. Similarly, plants of the invention may be transformed and regenerated, whereby one or more chimeric genes are introduced into the plants. Transformation can be carried out using standard methods, such as Agrobacterium tumefaciens mediated transformation or biolistics, followed by selection of the transformed cells and regeneration into plants. A desired trait (e.g. genes conferring pest or disease resistance, herbicide, fungicide or insecticide tolerance, etc.) can be introduced into the plants, or progeny thereof, by transforming a plant of the invention or progeny thereof with a transgene that confers the desired trait, wherein the transformed plant retains the chromosome comprising the mutant ms10 allele and the mutant aa allele, wherein the meiotic recombination frequency is reduced between said mutant ms10 allele and said mutant aa allele as described herein and, when the chromosome comprising the mutant ms10 allele and the mutant aa allele, wherein the meiotic recombination frequency is reduced between said mutant ms10 allele and said mutant aa allele is comprised in homozygous form, the male sterility and anthocyanin absent phenotype conferred by it and contains the desired trait.

In another embodiment the invention relates to a method for producing seed, comprising crossing a plant of the invention with itself or a different plant and harvesting the resulting seed. In a further embodiment the invention relates to seed produced according to this method and/or a plant produced by growing such seed. Thus, a plant of the invention may be used as male and/or female parent, in the production of seeds, whereby the plants grown from said seeds comprise the chromosome comprising the mutant ms10 allele and the mutant aa allele, wherein the meiotic recombination frequency is reduced between said mutant ms10 allele and said mutant aa allele as provided herewith.

Thus, in one aspect progeny of a Solanum lycopersicum plant of the invention are provided, wherein the progeny plant is produced by selfing, crossing, mutation, double haploid production or transformation and preferably wherein the progeny retain the chromosome comprising the mutant ms10 allele and the mutant aa allele, wherein the meiotic recombination frequency is reduced between said mutant ms10 allele and said mutant aa allele when compared to the meiotic recombination frequency between the MS10 gene and the AA gene in a wild type Solanum lycopersicum plant as provided herewith.

The present invention further provides a method of identifying and/or selecting a male sterile plant, said method comprising growing a plant according to the present invention and determining whether anthocyanin is absent in the hypocotyls of said plant.

The present invention further provides a method of identifying and/or selecting a plant of the present invention or part of a plant of the present invention. The present invention accordingly further provides a method of identifying and/or selecting a plant or plant part of the species Solanum lycopersicum comprising in its genome at least one chromosome comprising a mutant allele of the wild type MS10 gene and a mutant allele of the wild type AA gene wherein in said plant the meiotic recombination frequency is reduced between said mutant allele of the wild type MS10 gene and said mutant allele of the wild type AA gene when compared to the meiotic recombination frequency between the MS10 gene and the AA gene in a wild type Solanum lycopersicum plant, wherein the wild type MS10 gene encodes a protein comprising at least 95% amino acid sequence identity to SEQ ID NO: 1, e.g. 96%, 97%, 98%, 98.3%, 98.7%, 99.0%, or 99.3% or more preferably 99.7% sequence identity to SEQ ID NO: 1, (as determined using methods discloses elsewhere herein) and the mutant allele of the wild type MS10 gene results in no expression or reduced expression of the wild type gene and/or the mutant allele of the wild type MS10 gene encodes a protein having loss-of-function or reduced function when compared to the wild type protein, and wherein the wild type AA gene encodes a protein comprising at least 95% amino acid sequence identity to SEQ ID NO: 3, e.g. 96%, 97%, 98%, 98.3%, 98.7%, 99.0%, or 99.3% or more preferably 99.7% sequence identity to SEQ ID NO: 3, (as determined using methods discloses elsewhere herein) and the mutant allele of the wild type AA gene results in no expression or reduced expression of the wild type gene and/or the mutant allele of the wild type AA gene encodes a protein having loss-of-function or reduced function when compared to the wild type protein and wherein said method comprises determining whether the genomic DNA region between the MS10 gene and the AA gene has been modified resulting in that the meiotic recombination frequency between the MS10 gene and the AA gene is reduced when compared to the meiotic recombination frequency between the MS10 gene and the AA gene in a wild type Solanum lycopersicum plant.

As described herein, the plant or plant part of the species Solanum lycopersicum of the present invention and as used in the methods as described herein comprising in its genome at least one chromosome comprising a mutant allele of the wild type male sterility 10 (MS10) gene and a mutant allele of the wild type anthocyanin absent (AA) gene preferably comprises an inversion and/or a deletion in the genomic region between said mutant MS10 allele and said mutant AA allele.

The method comprises screening at the DNA, RNA (or cDNA) or protein level using known methods, in order to detect the presence of one or more of the mutant alleles according to the present invention and/or of the chromosome comprising the mutant ms10 allele and the mutant aa allele, wherein meiotic recombination is suppressed between said mutant ms10 allele and said mutant aa allele. There are many methods to detect the presence of a mutant allele of a gene.

For example, if there is a single nucleotide difference (single nucleotide polymorphism, SNP) between the wild type and the mutant allele, a SNP genotyping assay can be used to detect whether a plant or plant part or cell comprises the wild type nucleotide or the mutant nucleotide in its genome. For example, the SNP can easily be detected using a KASP-assay (see world wide web at kpbioscience.co.uk) or other SNP genotyping assays. For developing a KASP-assay, for example 70 base pairs upstream and 70 base pairs downstream of the SNP can be selected and two allele-specific forward primers and one allele specific reverse primer can be designed. See e.g. Allen et al. 2011, Plant Biotechnology J. 9, 1086-1099, especially p 097-1098 for KASP-assay method.

Equally other genotyping assays can be used. For example, a TaqMan SNP genotyping assay, a High Resolution Melting (HRM) assay, SNP-genotyping arrays (e.g. Fluidigm, Illumina, etc.) or DNA sequencing may equally be used.

Molecular markers may also be used to aid in the identification of the plants (or plant parts or nucleic acids obtained therefrom) containing the mutant ms10 allele and/or of the mutant aa allele and/or of the chromosome comprising the mutant ms10 allele and the mutant aa allele, wherein meiotic recombination is suppressed between said mutant ms10 allele and said mutant aa allele. For example, one can develop one or more suitable molecular markers which are closely genetically (and preferably also physically) linked to the mutant ms10 allele and/or the mutant aa allele and/or the chromosome comprising the mutant ms10 allele and the mutant aa allele, wherein meiotic recombination is suppressed between said mutant ms10 allele and said mutant aa allele. Most preferably, the causal gene mutation is used as the molecular marker used for the identification of the plants (or plant parts or nucleic acids obtained therefrom) containing the mutant ms10 allele and/or the mutant aa allele and/or the chromosome comprising the mutant ms10 allele and the mutant aa allele, wherein meiotic recombination is suppressed between said mutant ms10 allele and said mutant aa allele. Suitable molecular markers can be selected based on the available genetic information of the mutant plants. If no genetic information of the mutant plants is available, suitable molecular markers can be developed by crossing a Solanum lycopersicum plant according to the present invention (preferably having the male sterility trait reliably linked to the anthocyanin absent trait) with a wild type plant and developing a segregating population (e.g. F2 or backcross population) from that cross. The segregating population can then be phenotyped for the anthocyanin absent (or alternatively the male sterility) phenotype as described herein and genotyped using e.g. molecular markers such as SNPs (Single Nucleotide Polymorphisms), AFLPs (Amplified Fragment Length Polymorphisms; see, e.g., EP 534 858), or others, and by software analysis molecular markers which co-segregate with the traits of the present invention in the segregating population can be identified and their order and genetic distance (centimorgan distance, cM) to the MS10 gene (or locus) can be identified. Molecular markers which are closely linked to MS10 locus, e.g. markers at a 5 cM distance or less, can then be used in detecting and/or selecting plants (e.g. plants of the invention or progeny of a plant of the invention) or plant parts comprising or retaining the mutant ms10 allele (e.g. in an introgression fragment). Such closely linked molecular markers can replace phenotypic selection (or be used in addition to phenotypic selection) in breeding programs, i.e. in Marker Assisted Selection (MAS). Preferably, linked markers are used in MAS. More preferably, flanking markers are used in MAS, e.g. one marker on either side of the locus of the mutant ms10 allele.

The method of identifying and/or selecting a plant or plant part of the present invention accordingly comprises determining whether the genomic DNA region between the MS10 gene and the AA gene has been modified resulting in that the meiotic recombination frequency between the MS10 gene and the AA gene is reduced as described herein. As described herein above, there are several ways to provide a plant wherein the meiotic recombination frequency is reduced between two loci. The process step of determining whether the genomic DNA region between the MS10 gene and the AA gene has been modified to achieve the reduction of meiotic recombination frequency between the MS10 gene and the AA gene must accordingly be specifically selected. For instance, a deletion may be induced in the genome resulting in a reduction of the physical distance between the locus of the mutant allele of the wild type MS10 gene and the locus of the mutant allele of the wild type AA gene. It is accordingly determined whether the physical distance between the mutant MS10 gene and the mutant AA gene is reduced using standard methods well-known in the art. Alternatively, the meiotic recombination frequency between two loci can be suppressed by the introgression of an introgression fragment located between the two loci, wherein said introgression fragment has a sufficiently reduced homology compared to the wild type fragment to result in a reduction of the meiotic recombination frequency. It is accordingly determined whether a non-homologous introgression fragment is present between the mutant MS10 gene and the mutant AA gene using standard methods. Preferably, the meiotic recombination frequency between two loci is suppressed as the result of an inversion between said two loci in one of the chromosome pairs. It is accordingly determined whether an inversion is present between the mutant MS10 gene and the mutant AA gene using standard methods. The present invention accordingly preferably provides a method of identifying and/or selecting a plant or plant part of the species Solanum lycopersicum comprising in its genome at least one chromosome comprising a mutant allele of the wild type MS10 gene and a mutant allele of the wild type AA gene wherein in said plant the meiotic recombination frequency is reduced between said mutant allele of the wild type MS10 gene and said mutant allele of the wild type AA gene when compared to the meiotic recombination frequency between the MS10 gene and the AA gene in a wild type Solanum lycopersicum plant, wherein the wild type MS10 gene encodes a protein comprising at least 95% amino acid sequence identity to SEQ ID NO: 1, e.g. 96%, 97%, 98%, 98.3%, 98.7%, 99.0%, or 99.3% or more preferably 99.7% sequence identity to SEQ ID NO: 1, (as determined using methods discloses elsewhere herein) and the mutant allele of the wild type MS10 gene results in no expression or reduced expression of the wild type gene and/or the mutant allele of the wild type MS10 gene encodes a protein having loss-of-function or reduced function when compared to the wild type protein, and wherein the wild type AA gene encodes a protein comprising at least 95% amino acid sequence identity to SEQ ID NO: 3, e.g. 96%, 97%, 98%, 98.3%, 98.7%, 99.0%, or 99.3% or more preferably 99.7% sequence identity to SEQ ID NO: 3, (as determined using methods discloses elsewhere herein) and the mutant allele of the wild type AA gene results in no expression or reduced expression of the wild type gene and/or the mutant allele of the wild type AA gene encodes a protein having loss-of-function or reduced function when compared to the wild type protein and wherein said method comprises determining whether the genomic DNA region between the MS10 gene and the AA gene comprises an inversion resulting in that the meiotic recombination frequency between the MS10 gene and the AA gene is reduced when compared to the meiotic recombination frequency between the MS10 gene and the AA gene in a wild type Solanum lycopersicum plant.

Preferably, the method of identifying and/or selecting a plant or plant part of the species Solanum lycopersicum according to the present invention comprises a preceding process step wherein a double strand break is induced in or near the wild type MS10 gene to provide the mutant allele of the wild type MS10 gene and/or a double strand break is induced in or near the wild type AA gene to provide the mutant allele of the wild type aa gene prior to determining whether the genomic DNA region between the MS10 gene and the AA gene has been modified resulting in that the meiotic recombination frequency between the MS10 gene and the AA gene is reduced. Said double strand break inducing step may comprise contacting said plant or plant part with an engineered nuclease upon which said double strand break may be repaired by the cell's endogenous DNA double stranded break repair mechanisms (e.g. the homology directed repair mechanism), which allows a site-specific deletion or inversion of DNA in a target cell. Engineered nucleases useful in genome editing methods include meganucleases, zinc finger nucleases (ZFNs), transcription activator-like effector-based nucleases (TALEN), and clustered regularly interspaced short palindromic repeats (CRISPR)-associated nucleases. Genome editing methods particularly useful in the context of the present invention include, but are not limited to, CRISPR/Cas9-based targeted mutagenesis methods and CRISPR/Cas12 (also known as CRISPR/Cpf1)-based targeted mutagenesis methods; see e.g. Brooks et al. (2014) Plant Physiol 166, 1292-1297 and WO2016/205711 A1.

The process step of determining whether the genomic DNA region between the MS10 gene and the AA gene has been modified resulting in that the meiotic recombination frequency between the MS10 gene and the AA gene is reduced preferably comprises determining whether the plant or plant part comprises an inversion or a deletion of a genomic DNA fragment located between the MS10 gene and the AA gene.

Preferably, the method of identifying and/or selecting a plant or plant part of the species Solanum lycopersicum according to the present invention comprises a preceding process step wherein a double strand break is induced in or near the wild type MS10 gene to provide the mutant allele of the wild type MS10 gene and wherein a double strand break is induced in or near the wild type AA gene to provide the mutant allele of the wild type aa gene prior to determining whether the genomic DNA region between the MS10 gene and the AA gene has been modified resulting in that the meiotic recombination frequency between the MS10 gene and the AA gene is reduced.

The present invention further provides a method for producing a plant or plant part having male sterility and anthocyanin absent hypocotyls, wherein in said plant or plant part the meiotic recombination frequency between the male sterility trait and the anthocyanin absent hypocotyls trait is reduced, said method comprising: (a) inducing in a plant or plant part a double strand break in both the MS10 gene and the AA gene, wherein the wild type MS10 gene encodes a protein comprising at least 95% amino acid sequence identity to SEQ ID NO: 1 and wherein the wild type AA gene encodes a protein comprising at least 95% amino acid sequence identity to SEQ ID NO: 3; (b) optionally regenerating the plant part in which the double strand break is induced into a plant or into a different plant part. In the method for producing a plant or plant part having male sterility and anthocyanin absent hypocotyls the double strand break in both the MS10 gene and the AA gene are preferably induced using an engineered endonuclease. Preferably, the double strand break is induced using an engineered endonuclease, wherein said engineered endonuclease preferably is a meganuclease, zinc finger nuclease (ZFN), transcription activator-like effector-based nuclease (TALEN) or a clustered regularly interspaced short palindromic repeats (CRISPR)-associated nuclease.

As described, inducing a double strand break in both the MS10 gene and the AA gene may induce a deletion of a genomic DNA fragment located between the double strand breaks or may induce an inversion of the genomic DNA fragment located between the double strand breaks In the context of the present invention, however, the double strand break in both the MS10 gene and the AA gene preferably induces an inversion and/or a deletion of the genomic DNA fragment located between the double strand breaks. An inversion is preferred over a deletion since said deletion may lead to additional undesired characteristics in the event said deletion leads to the loss of protein function of other genes than the MS10 gene and the AA gene.

Known methods for inducing a double strand break using an engineered endonuclease requires that a plant or plant part is (transiently) transformed. Preferably, the double strand break is induced in a protoplast, callus or microspore. Transformation can be carried out using standard methods, such as Agrobacterium tumefaciens mediated transformation or biolistics, followed by selection of the transformed cells and regeneration into plants.

The double strand breaks induced in the method for producing a plant or plant part having male sterility and anthocyanin absent hypocotyls according to the present invention preferably result in the mutant allele of the wild type male MS10 gene and the mutant allele of the wild type AA gene as described in more detail herein above and result in the suppression of meiotic recombination between said mutant allele of the wild type MS10 gene and said mutant allele of the wild type AA gene as described herein, for instance by inducing a deletion or an inversion. Accordingly, the double strand break induced in the wild type MS10 gene preferably leads to no expression or reduced expression of the MS10 gene and/or a loss-of-function or reduced function of the protein encoded by said MS10 gene; and/or the double strand break induced in the wild type AA gene preferably leads to no expression or reduced expression of the AA gene and/or to a loss-of-function or reduced function of the protein encoded by said AA gene. Even more preferably, the double strand break induced in the wild type MS10 gene leads to no expression of the MS10 gene and/or a loss-of-function of the protein encoded by said MS10 gene; and/or the double strand break induced in the wild type AA gene leads to no expression of the AA gene and/or to a loss-of-function of the protein encoded by said AA gene.

In one aspect plants, plant parts and cells according to the invention are not exclusively obtained by means of an essentially biological process as defined by Rule 28(2) EPC.

### EXAMPLES

### Example 1

### Designing and Cloning of the Constructs

For design of gRNAs for MS, we used the first exon of MS. For design of gRNAs for AA, we used the first and second exon of this gene. For each gene we designed two gRNAs (Table 1), using the software CRISPOR (http://crispor.tefor.net).

TABLE 1
gRNAs for making double strands DNA breaks in the male sterility gene MS and
the anthocyanin absent gene AA, both on Ch02.

| Targeted gene | name | gRNA | Position SL3.0ch02 |
|---|---|---|---|
| MS | gMS1 | GGCGTCAAAAACTTAGCGAA AGG (SEQ ID NO: 5) | 44796492 |
| MS | gMS2 | ATACAAATCCAAGAACCTTA AGG (SEQ ID NO: 6) | 44796528 |
| AA | gAA1 | GAAAGTGTATGGTTCAGCAA TGG (SEQ ID NO: 7) | 45896386 |
| AA | gAA2 | CAATGGCTGCATGTCCACAA AGG (SEQ ID NO: 8) | 45896403 |

Two CRISPR-Cas constructs were made, combining one gRNA for AA with one for MS. Construct 1 contained gMS1 and gAA1, Construct 2 harbored gMS2 and gAA2.

The CRISPR-Cas constructs were built, by means of cloning using the Golden Gate Mo-Clo Toolkit (https://www.addgene.org/kits/marillonnet-moclo/). The designed gRNAs were ordered as oligos and each one was inserted separately behind the U6-26 promotor from Arabidopsis thaliana (pICSL90002 plasmid, https://www.addgene.org/68261/). The gRNAs with promotor were combined with the Arabidopsis codon-optimized SpCas9 sequence under the Petroselinum crispum Ubiquitin4-2 promoter and NOS terminator (pDe-CAS9, https://www.addgene.org/61433/). We included in the CRISPR-Cas construct a fluorescence GFP gene (p35S-fGFP-ter35S), driven by CaMV-35S promotor and terminator, for estimation of the proportion of successfully transfected protoplasts.

### Plasmid Isolation, Harboring CRISPR-Cas Construct 1 or 2

The plasmids harboring the two CRISPR-Cas constructs were propagated in Escherichia coli (25 ml LB) and isolated, using the QIAGEN plasmid midi kit (Cat No./ID: 12143), as described in the manual.

The plasmid DNA was eluted in 200 μl EB buffer and quantified using the Nanodrop One (Thermofisher). For the transfection, bug plasmid was pipetted in a 2 ml tube, and water was added till the volume reached 20 μl.

### Growing Plants

Tomato seeds cv. Moneyberg were sterilised in 1% bleach for 20 minutes (room temperature). After sterilization seeds were washed in MilliQ water (2 minutes room temperature). Seeds were sown on germination medium (½ MS including vitamins (Duchefa), 1% sucrose and 0.8% Daishin agar, pH5.8) in sterile tissue culture vessels (OS140BOX/green filter, Duchefa), 4 seeds/vessel. Plants were grown under long day conditions.

### Protoplast Isolation and Washing

2-3 months old plants were used as starting material for protoplast isolation. Multiple (4-5) Leaves were cut off using scalpel and tweezers and moved to a 9 cm petri dish containing 10 ml digestion buffer (0.4 M mannitol, 20 mM MES, 20 mM KCL and 10 mM CaCl2), pH5.7). Leaves were cut in a feather-like pattern, from the midrib to the edge of the leaves (scalpel blade 11). This was repeated until the whole surface of the petri-dish was covered (approximately 15-20 leaves), usually enough for 25 transfections. After cutting the leaves, the digestion buffer was removed using a serological pipette and 10 ml of (freshly prepared) digestion buffer with 0.25% macerozyme R10 (2.5 g/L, M8002 Duchefa Biochemie) and 1% cellulase R10 (10 g/L, C8001 Duchefa Biochemie) was added to the petri dish. The plates were incubated in the dark for 16-18 hrs at 25° C.

Before starting the washing and transfection procedure PEG solution was prepared by mixing 4.0 g PEG-4000 (Fluke), 3.0 mL MilliQ, 2.5 mL 0.8 M mannitol solution and 1.0 mL 1 M CaCl2) solution in a 50 mL tube (and put it on a rollerbank).

After this incubation the plate was gently swirled horizontally by hand (about 30 times) to release the protoplasts. Using a 25 ml serological pipette the protoplast suspension was gently transferred through a Falcon 100 μm cell strainer (Corning) and carefully collected into a 50 ml tube. 10 ml of W5 washing buffer (154 mM NaCl, 125 mM CaCl2.2H2O, 5 mM KCL and 2 mM MES, pH5.7) was added to the petri dish and the dish was shaken 30 times to release additional protoplasts. This suspension was again transferred through the cell strainer to the same 50 ml tube. The tube was centrifuged at 100×g for 3 minutes at room temperature to pellet the protoplasts (break and acceleration of the centrifuge were set at 6, Eppendorf 5810R). The supernatant was quickly poured off and 10 ml of W5 buffer was added to the tube (pipetted against the wall of the tube). Protoplasts were resuspended carefully by slowly turning the tube. This washing step was repeated once. After the second wash protoplasts were resuspended in 10 ml MMg solution (0.4 M Mannitol, 15 mM MgCl2 and 4 mM MES, pH5.7), centrifuged 3 minutes at 100×g at room temperature, and resuspended in 10 ml MMg solution. Protoplasts were counted using a haemocytometer and diluted with MMg solution to a density of 1 million protoplasts per ml.

### Protoplast Transfection

Per transfection, 10 μg of plasmid DNA solution (in 5 mM Tris or MilliQ) was added to a 2 ml tube (total volume adjusted 20 μl with MilliQ). 200 μl of protoplast suspension was added (and not yet mixed) to each tube (using wide-orifice tips). After this 200 μl of PEG solution (freshly prepared, see above) was added to the first tube and mixed by carefully and repeatedly inverting (until mixture was homogeneous) before continuing to the next tube. The mixtures were incubated for 10-20 minutes (10 minutes is enough but with multiple samples more time is needed). 500 μl of W1 buffer (0.5 M Mannitol, 20 mM KCl and 4 mM MES, pH5.7) was added droplet-wise to the first sample and then mixed carefully before continuing to the next sample etc. After the last sample this step was repeated so that a total of 1 ml of W1 buffer was added to all the samples. Tubes were centrifuged at 200×g for 3 minutes at room temperature and the supernatant was carefully removed by pipetting. Another 1 ml W1 buffer was added to each tube (like in the previous step). Samples were centrifuged again and finally the protoplasts were resuspended in 150 μl W1 buffer. Protoplasts were incubated for 24 hours at 25° C. (in the dark) before further analysis.

### Checking Presence of Induced Inversions

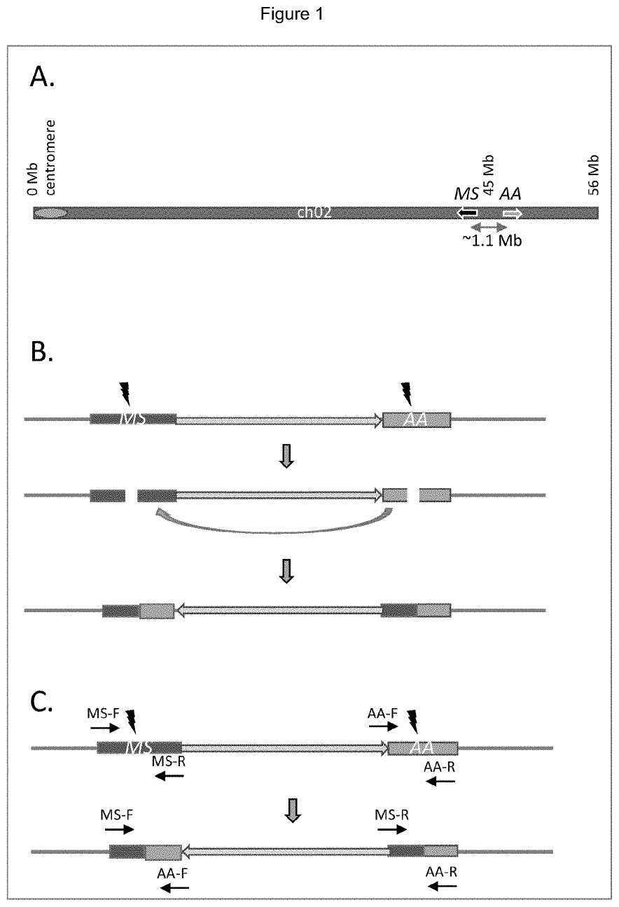

For checking for presence of induced mutations, we used targeted PCRs, as shown in FIGS. 1B and C. Forward and reverse primers were designed around the MS and AA guides, using the reference genome for sequences flanking the gRNA loci, and primer design software Primer3 (http://bioinfo.ut.ee/primer3-0.4.0/). The primers pairs are (AA-F) 5′-TGGTTGCTGCTCATCTTCAC-3′ (SEQ ID NO: 9) with (AA-R) 5′-GCAAAGCCACCTTCATTCAT-3′ (SEQ ID NO: 10) and (MS-F) 5′-TAGGGGATTTTCATGCTGGT-3′ (SEQ ID NO: 11) with (MS-R) 5′-GCCAAAAATGAGTCCTTCCA-3′ (SEQ ID NO: 12).

The transfected protoplasts were processed in the following way: Per sample, 2 μl of the isolated protoplasts were diluted 1:1 with a 20 mM KOH, 1% caseine solution, boiled for 5 minutes, and put on ice for 5 minutes.

The left-hand side and the ‘right-hand side’ of the induced inversions were amplified by means of PCR, using Phire polymerase (Thermofisher). For the left end, the forward MS primer (MS-F) and forward AA primer (AA-F) were used, while for the right-hand side we used the reverse MS-R and reverse AA-R primers (FIG. 1C). The reaction mix contained 5 μl 5× Buffer, 1 μl 5 mM DNTP, 1.25 μl 10 pmol/μl F primer, 10 pmol/μl R primer, 0.35 μl Phire polymerase, 4 μl protoplast solution (˜2700 protoplasts), and 12.5 μl water. A water control was also included. Eighty PCR cycles (10 sec at 98° C., 20 sec at 61.5° C., and 40 sec extension time at 72° C.) were performed to detect also rarely occurring events. As a negative control, non-transfected protoplasts were used. PCR products were brought on agarose gel to visualize them.

qPCR

To estimate the frequency of the inversion events, a qPCR was performed on the processed protoplast samples. This was done with the iQ SYBR Green Supermix (Biorad) on the qPCR machine (Biorad). Reaction conditions were as follows: 12.5 μl iQ SYBR Green Supermix, 1.25 μl 10 pmol/μl F primer, 10 pmol/μl R primer, 2 μl protoplast solution (˜1300 protoplasts), water till 25 μl.

As reference the Actin gene from tomato was included, using as forward and reverse primer sequences 5′-ACTGTCCCTATCTATGAAGGTTATGC-3′ (SEQ ID NO: 13) and 5′-GAAACAGACAGGACACTCGCACT-3′ (SEQ ID NO: 14), respectively.

### Plant Transformation

Transgenic plants (TO) containing the CRISPR-Cas were generated by Agrobacterium tumefaciens-mediated transformation. The following transformation method may accordingly be used. Cotyledons of 10-day-old seedlings are incubated in 8 ml Agrobacterium cells suspended in 2% MSO (liquid MS medium, containing 100 mg L⁻¹ myo-inositol, 400 μg L⁻¹ thiamine HCl and 20 g L⁻¹ sucrose) to an attenuance at 600 nm of 0.5. After 30 min, the cotyledons are blotted dry on sterile filter paper and placed on MS culture medium containing 1× Nitsch and Nitsch vitamin mixture, 3% w/v sucrose, 1 mg L⁻¹ NAA, 1 mg L⁻¹ 6-benzylaminopurine and 0.7% w/v agar, pH 5.7. After 2 days of co-cultivation, the cotyledons are washed in liquid MS medium with 200 mg L⁻¹ carbenicillin and transferred to shoot-inducing MS culture medium containing 1× Nitsch and Nitsch vitamin mixture, 3% w/v sucrose, 2 mg L⁻¹ zeatin, 200 mg L⁻¹ carbenicillin, 0.7% w/v agar, pH 5.7 and 100 mg L⁻¹ kanamycin for selection. Cotyledons that start to develop callus are transferred to fresh culture medium, containing half of the zeatin concentration and 1 mg L⁻¹ GA3. The cotyledons are transferred to fresh medium every 2 weeks. When initial calli formed, shoot primordia are excised and transferred to shoot-elongation MS culture medium, which is germination medium containing 200 mg L⁻¹ carbenicillin and 100 mg L⁻¹ kanamycin. Elongated shoots of 2-4 cm were excised from the callus and transferred to rooting MS culture medium (1× Nitsch and Nitsch vitamin mixture, 1.5% w/v sucrose, 5 mg L⁻¹ IAA, 200 mg L⁻¹ carbenicillin, 50 mg L⁻¹ kanamycin and 0.7% w/v agar, pH 5.7). Rooted (TO) plantlets are transferred to soil for further analysis. Media components and antibiotics are obtained from Duchefa Biochemie.

### Selection of T0 Transgenic Plant Containing an Active the CRISPR-Cas Construct

The activity of the CRISPR-Cas construct is dependent on the location of the integration in the genome. To identify T0 plants with an active construct plants containing mutations were identified by sequencing the targeted regions in the MS and AA genes in the T0 plants. These domains were amplified by means of PCR, using Phire polymerase (Thermofisher). Primer set specific for either the targeted region in the MS or the AA genes were used. The reaction mix contained 5 μl 5× Buffer, 1 μl 5 mM DNTP, 1.25 μl 10 pmol/μl forward primer, 10 pmol/μl reverse primer, 0.35 μl Phire polymerase, 4 μl genomic DNA solution (4 ng), and 12.5 μl water. Thirty PCR cycles (10 sec at 98° C., 20 sec at 60° C., and 5 sec extension time at 72° C.) were performed to amplify the target regions. PCR products were sequenced by a service provider and the presence of mutations determined using a computer program for DNA sequence analysis.

Self-Pollination and Crossing TO Plants with Plants not Comprising Said Exogenous DNA to Provide a Plurality of Progeny Plants

Selected T0 plants with an active CRISPR-Cas construct were grown and flowers were allowed to self-pollination to produce fruits with T1 seeds. Fruits were grown an T1 seeds were collected.

Alternatively, selected T0 plants with an active CRISPR-Cas construct were grown and flowers were emasculated before anther dehiscence to prevent self-pollination. Pollen, isolated from a wild type plant were collected and used to pollinate selected T0 plants, fruits were grown and F1 seeds were collected. F1 plants containing an active CRISPR-Cas construct can be grown to produce F2 seeds.

T1, F1 and F2 seeds were germinated and genomic DNA isolated from first leaves. PCRs were designed to detect desired mutations, inversions or other structural variations. By using a pooling strategy (e.g. Tsai et al., 2011; https://doi.org/10.1104/pp. 110.169748) large number of seedlings can be analyzed with only limited number of PCRs.

### Example 2

CRISPR-Cas9 constructs were built as described in Example 1. Three CRISPR-Cas9 constructs were made, combining one gRNA for AA with one for MS. Construct 1 contained gMS1 and gAA1, construct 3 gMS3 and gAA3, and construct 4 gMS4 and gAA4.

TABLE 2
gRNA target sites for making double-strand DNA breaks in the male sterility
gene MS and the anthocyanin absent gene AA, both on Ch02 of tomato. The ⬇ sign is
placed at the location where the double-strand break is expected to take place. The
PAM site is displayed in italics.

| Targeted gene | name | gRNA | Position SL3.0ch02* |
|---|---|---|---|
| MS | gMS1 | GGCGTCAAAAACTTAGC⬇GAA AGG (SEQ ID | 44796509-44796487 |
| | | NO: 5) | |
| MS | gMS3 | AACTCTGAAGAAAGGGA⬇AGT AGG (SEQ ID | 44796612-44796590 |
| | | NO: 15) | |
| MS | gMS4 | ATTCAAACAACTCTGAA⬇GAA AGG (SEQ ID | 44796620-44796598 |
| | | NO: 16) | |
| AA | gAA1 | GAAAGTGTATGGTTCAG⬇CAA TGG (SEQ ID | 45896370-45896392 |
| | | NO: 7) | |
| AA | gAA3 | AATGGCTGCATGTCCAC⬇AAA GGG (SEQ ID | 45896388-45896410 |
| | | NO: 17) | |
| AA | gAA4 | ATGGTTTGTCTTATAGA⬇ATT GGG (SEQ ID | 45896413-45896435 |
| | | NO: 18) | |

*Solanum lycopersicum cultivar Heinz 1706 chromosome 2, SL3.0.

The constructs were propagated in Escherichia coli, and plasmids were isolated and purified for transfection of tomato protoplasts. Protoplasts were isolated from in vitro grown plant leaves of ‘Moneymaker’. After transfection and incubation, the cell cultures were screened for inversions by PCR. To do that a part of the transfected protoplasts (˜2.700 cells) were transferred to a PCR tube and denatured in KOH as described in Example 1.

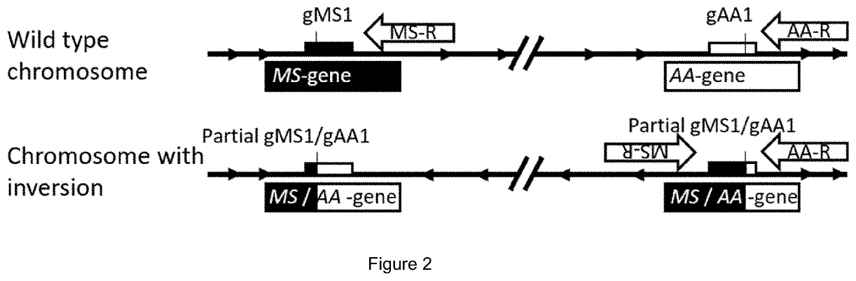

A PCR was performed on the cell lysate using primer combinations pairs AA-F with MS-F or AA-R with MS-R in separate PCRs. The annealing strand of one pair of primers is the same, preventing amplification on wild type DNA, and allowing amplification only in case of an induces inversion (FIG. 2). In addition, the distance between the primers of one pair is ˜1.1 Mbp on the wild type genome, which too large to amplify a PCR product.

TABLE 3
PCR primers used to amplify the borders of the inversion

| Primer name | Primer sequence | Position SL3.0ch02* |
|---|---|---|
| AA-F | 5′-TGGTTGCTGCTCATCTTCAC-3′ (SEQ ID NO: 9) | 43326933-43326952 |
| MS-F | 5′-TAGGGGATTTTCATGCTGGT-3′ (SEQ ID NO: 11) | 42223166-42223185 |
| AA-R | 5′-GCAAAGCCACCTTCATTCAT-3′ (SEQ ID NO: 10) | 43328249-43328230 |
| MS-R | 5′-GCCAAAAATGAGTCCTTCCA-3′ (SEQ ID NO: 12) | 42224500-42224481 |

*Solanum lycopersicum cultivar Heinz 1706 chromosome 2, SL3.0.

However, an inversion of the region between the gRNA targets, would bring together the primer sites in an orientation that would make amplification of a PCR product possible, including the border of the inversion as depicted in FIG. 2.

PCR products from protoplasts transfected with construct 1, 3, and 4 were separated on an agarose gel and fragments with the expected size were excised from gel and the DNA was Sanger sequenced. It was surprising that in most PCRs DNA fragments the expected size were generated, which would mean that inversions take place in one or more protoplasts per reaction. Considering that one reaction contains the DNA from about 2700 protoplasts, and that the transfection efficiency was about 70% (appearing from fluorescence of the GFP gene, present in the constructs too; data not shown), that would mean that the inversion frequency is >1/(0.7*2700) cells. The expected PCR amplicon sizes were based on the location of the primer and gRNA binding sites on the genome, which is about 1.3 kb. The primer and gRNAs binding site locations and their sequences are depicted in tables 2 and 3.

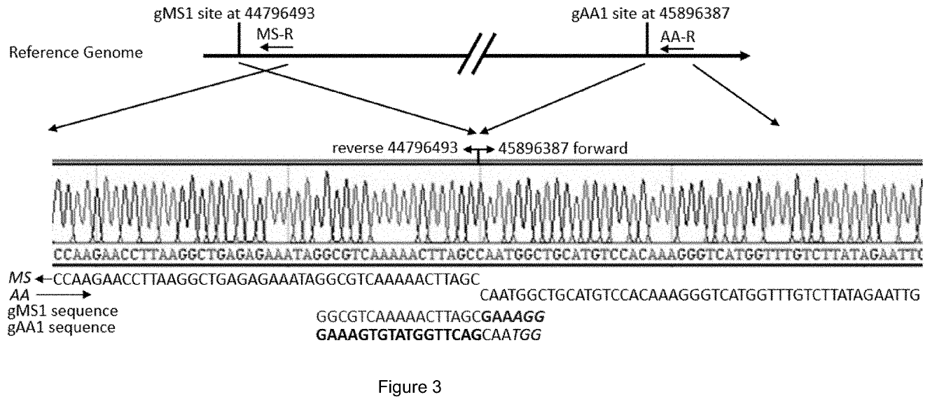

FIG. 3 shows a part of the PCR product's DNA sequence generated with the primers MS-R and AA-R, and genomic DNA from a protoplast culture transfected with construct 1, harboring the gRNAs gMS1 and gAA1. The sequence is from the downstream right end of the inversion. The alignment in the lower part of the figure shows that the DNA had been cleaved in the gMS1 binding site, and that the upstream part has been linked to the gAA1 binding site. Apparently, the Cas enzyme had generated double-strand breaks (DSBs) at both gRNA binding sites, leading to the inversion of the ˜1.1 Mbp chromosome fragment in between. The DSB were generated at exactly the predicted locations in the gRNA binding sites, which is between three and four basepairs upstream the PAM site. The ligation of the ends of the inverted chromosome fragment had been made without any additional sequence modification.

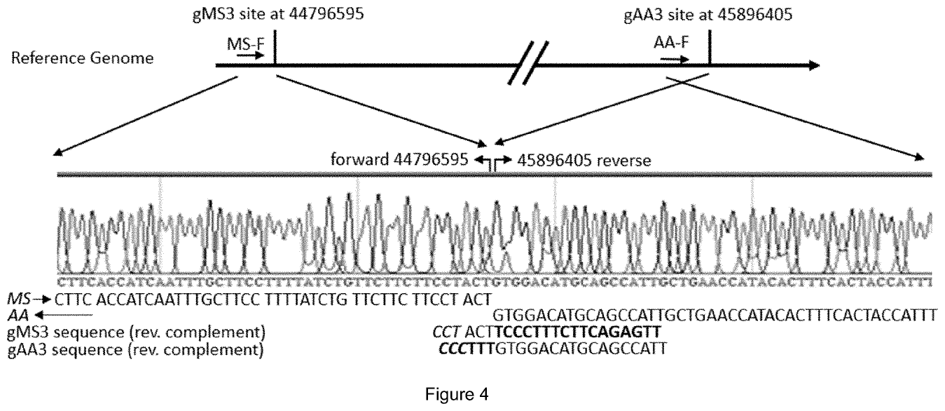

FIG. 4 shows part of the PCR product's DNA sequence generated with primers MS-F and AA-F and genomic DNA from protoplasts transfected with construct 3. The sequence is from the upstream left end of the inversion. The alignment in the lower part of the figure shows that the DNA had been cleaved at the gMS3 binding site and that the upstream part has been linked to the gAA3 binding site.

The Cas9 enzyme also here generated double-strand breaks (DSBs) at both gRNA binding sites, leading to the inversion of the ˜1.1 Mbp chromosome fragment in between. The DSBs were generated at exactly the predicted locations in the gRNA binding sites, and the ligation of the ends of the inverted chromosome fragment had been made without any additional sequence modification.

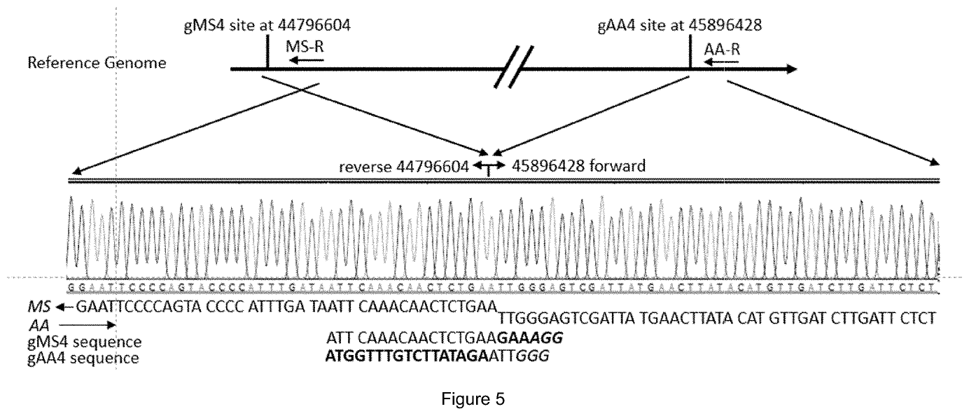

FIG. 5 shows part of the PCR product's DNA sequence generated with primers MS-R and AA-R and genomic DNA from protoplasts transfected with construct 4. The sequence is the downstream right end of the inversion. The alignment in the lower part of the figure shows that the DNA had been cleaved at the location of the gMS4 binding site, and that it has been linked to the location of gAA4 binding site. The Cas enzyme had also here generated the DSBs at both sides of the induced inversion at exactly the predicted location in the gRNA binding sites, and the ligation of the ends has been made with a deletion of one adenosine at the ligation site.

The three sequences in the FIGS. 3 to 5 show that the gRNAs target the Cas9 to the predicted positions and that DNA cleavage takes place at the expected position. In addition, the sequenced transitions demonstrate that DSBs had been generated at two locations on the chromosome and that in some cases the chromosome fragment in-between had been inversed after repair. The sequences of the ligated ends (the transition) demonstrate that the DSBs were generated at the predicted positions.

Example 2 accordingly shows that inversions can be detected by amplification of the borders from the inversion in most of the PCRs containing the DNA from ˜2700 protoplasts. As explained above this would mean that the inversion takes place in about 1 of 2700*0.7 (˜transfection efficiency)=˜1900 protoplasts. To obtain a plant with the desired mutation would require a large number (>1900) regenerated shoots to have a chance on finding one with the desired mutation. In many species, including tomato, regeneration of shoots from protoplasts is technically very difficult, and has in some species never successfully been applied.

To solve this technical problem, the present invention provides a seed-based screening approach to identify such mutation. In general, the CRISPR-Cas construct causes double strand breaks in the DNA, that in the majority of events are repaired. The repair system often modifies the guide-RNA (gRNA) binding site, meaning that after repair the binding site for the gRNA is gone and no new double strand breaks can be generated. However, when a plant containing an active CRISPR-Cas construct is crossed with a wild type plant, then at least half of the seeds produced will contain an active CRISPR-Cas construct in addition to wild type DNA with unmodified gRNA binding sites. This means that each seed from such plant provides a new chance of inducing and identification of the seldom occurring mutation event. Thus, if an event, e.g. an inversion, occurs in 1 per 1900 events, then screening a multiplicity of this number of seedlings can be done to identify the desired mutation. The screening could be a PCR with border specific primers as describes above for protoplasts. To reduce the number of individual PCRs, a pooling strategy, one-, two- or three-dimensional may be designed.

The herein described method accordingly for the first time enables the provision of tomato plants comprising three desirable traits at the same time:
1. Knocking out MS, leading to male sterility in homozygous plants, facilitating production of hybrid seeds;
2. Knocking out AA, leading to absence of anthocyanin, and therefore loss of purple hypocotyl color;
3. Genetic linkage of these two traits, because of suppression of meiotic recombination between the mutant alleles ms and aa caused by the inversion, and therefore loss of homology of the sequence between the two genes.

1. A plant of the species Solanum lycopersicum comprising in its genome at least one chromosome comprising a mutant allele of the wild type male sterility 10 (MS10) gene and a mutant allele of the wild type anthocyanin absent (AA) gene wherein in said plant the meiotic recombination frequency is reduced between said mutant allele of the wild type MS10 gene and said mutant allele of the wild type AA gene when compared to the meiotic recombination frequency between the MS10 gene and the AA gene in a wild type Solanum lycopersicum plant,
wherein the wild type MS10 gene encodes a protein comprising at least 95% amino acid sequence identity to SEQ ID NO: 1 and the mutant allele of the wild type MS10 gene results in no expression or reduced expression of the wild type gene and/or the mutant allele of the wild type MS10 gene encodes a protein having loss-of-function or reduced function when compared to the wild type protein, and
wherein the wild type AA gene encodes a protein comprising at least 95% amino acid sequence identity to SEQ ID NO: 3 and the mutant allele of the wild type AA gene results in no expression or reduced expression of the wild type gene and/or the mutant allele of the wild type AA gene encodes a protein having loss-of-function or reduced function when compared to the wild type protein.

2. The plant according to claim 1, wherein the plant is homozygous for the mutant allele of the wild type male sterility 10 (MS10) gene and homozygous for the mutant allele of the wild type anthocyanin absent (AA) gene.

3. The plant according to claim 1, wherein the mutant allele of the wild type male MS10 gene (mutant ms10 allele) when present in homozygous form induces male sterility.

4. The plant according to claim 1, wherein the mutant allele of the wild type AA gene (mutant aa allele) when present in homozygous form induces the absence of anthocyanin in the hypocotyls.

5. The plant according to claim 1, wherein the chromosome comprising a mutant allele of the wild type male sterility 10 gene (mutant ms10 allele) and a mutant allele of the wild type anthocyanin absent gene (mutant aa allele) comprises an inversion and/or a deletion in the genomic region between said mutant ms10 allele and said mutant aa allele.

6. The plant according to claim 1, wherein the reduced meiotic recombination frequency corresponds to a genetic distance between the mutant ms10 allele and the mutant aa allele of less than 6 cM.

7. The plant according to claim 1, wherein the plant is an inbred plant, a dihaploid plant or a hybrid plant.

8. A seed from which a plant according to claim 1 can be grown.

9. A part of the plant according to claim 1, wherein said plant part is a leaf, anther, pistil, stem, petiole, root, ovule, pollen, microspore, protoplast, callus, tissue, seed, flower, cotyledon, hypocotyl, embryo or cell.

10. A method of identifying and/or selecting a male sterile plant, said method comprising growing a plant according to claim 1 and determining whether anthocyanin is absent in the hypocotyls of said plant.

11. A method of identifying and/or selecting a plant or plant part of the species Solanum lycopersicum comprising in its genome at least one chromosome comprising a mutant allele of the wild type MS10 gene and a mutant allele of the wild type AA gene,
wherein the wild type MS10 gene encodes a protein comprising at least 95% amino acid sequence identity to SEQ ID NO: 1 and the mutant allele of the wild type MS10 gene results in no expression or reduced expression of the wild type gene and/or the mutant allele of the wild type MS10 gene encodes a protein having loss-of-function or reduced function when compared to the wild type protein, and
wherein the wild type AA gene encodes a protein comprising at least 95% amino acid sequence identity to SEQ ID NO: 3 and the mutant allele of the wild type AA gene results in no expression or reduced expression of the wild type gene and/or the mutant allele of the wild type AA gene encodes a protein having loss-of-function or reduced function when compared to the wild type protein, and
wherein said method comprises determining whether the genomic DNA region between the MS10 gene and the AA gene has been modified resulting in that the meiotic recombination frequency between the MS10 gene and the AA gene is reduced when compared to the meiotic recombination frequency between the MS10 gene and the AA gene in a wild type Solanum lycopersicum plant.

12. The method according to claim 11, wherein the plant or plant part is subjected to a step wherein a double strand break is induced in or near the wild type MS10 gene to provide the mutant allele of the wild type MS10 gene and/or a double strand break is induced in or near the wild type AA gene to provide the mutant allele of the wild type AA gene prior to determining whether the genomic DNA region between the MS10 gene and the AA gene has been modified resulting in that meiotic recombination frequency between the MS10 gene and the AA gene is reduced when compared to the meiotic recombination frequency between the MS10 gene and the AA gene in a wild type Solanum lycopersicum plant.

13. A method for producing a plant or plant part of the species Solanum lycopersicum having male sterility and anthocyanin absent hypocotyls, wherein in said plant or plant part meiotic recombination frequency between the male sterility trait and the anthocyanin absent hypocotyls trait is reduced when compared to the meiotic recombination frequency between the MS10 gene and the AA gene in a wild type Solanum lycopersicum plant, said method comprising:
(a) inducing in a plant or plant part a double strand break in both the MS10 gene and the AA gene, wherein the wild type MS10 gene encodes a protein comprising at least 95% amino acid sequence identity to SEQ ID NO: 1 and wherein the wild type AA gene encodes a protein comprising at least 95% amino acid sequence identity to SEQ ID NO: 3; and
(b) optionally regenerating the plant part in which the double strand break is induced into a plant or into a different plant part.

14. The method according to claim 13, wherein the double strand break in both the MS10 gene and the AA gene induces an inversion and/or a deletion of the genomic DNA fragment located between the double strand breaks.

15. The method according to claim 13, wherein the double strand break is induced in a protoplast, callus or microspore.

16. The method according to claim 13, wherein the double strand break induced in the wild type MS10 gene leads to no expression or reduced expression of the MS10 gene and/or a loss-of-function or reduced function of the protein encoded by said MS10 gene; and/or wherein the double strand break induced in the wild type AA gene leads to no expression or reduced expression of the AA gene and/or to a loss-of-function or reduced function of the protein encoded by said AA gene.

17. The method according to claim 13, wherein the double strand break is induced using an engineered endonuclease.

18. The method according of claim 13, wherein said engineered endonuclease is a meganuclease, zinc finger nuclease (ZFN), transcription activator-like effector-based nuclease (TALEN) or a clustered regularly interspaced short palindromic repeats (CRISPR)-associated nuclease.


SEQUENCE LISTING (29 sequences)

SEQ ID NO: 1 - protein, 209 aa, Solanum lycopersicum
MEFPSTPFDNSNNSEEREVGRRTDKRKQIDGEVKEYKSKNLKAERNRRQKLSERLLQLRSLVPNITNMTKETIITDAITYIRELQMNVDNLSEQLLEMEATQGEELETKNEEIIDTADEMGKWGIEPEVQVANIGPTKLWIKIVCQKKRGGLTKLMEAMNALGFDINDTSATASKGAILITSSVEVVRGGLTEANRIREILLEIIHGIY

SEQ ID NO: 2 - DNA, 2696 bp, Solanum lycopersicum
tgtaaaaaagaaaaagtgaaaggagaataaaatttctacttaaagcagctctggtcactcatttgtactttacaatgccagtaatactagtaacaaccacaaagtagtcacaaacataactagcaagaacattacccacttctattcttctttctgtttgattccttttctttcgggtggttgttactctttaaccctatagccttttacgagtcctcataacggaggtgggttttattaagttgacaactaggcgtaaataatcaaaccatgaggattccaactattatgcatactagttggttggcgatgtctatattcaatcattgttatgctatatgcatactagttagtatgtaaacataaaaaaagtacttgttcaacttagtcccaaacaagttaaactcggggtatatgaatatttactgaccatatttcccgtttgaatttatctctttcgggataacaataacagtgaaaagtgtaattagaccaaaactgaaaagggttgtagtgtctgaacctacaaaaaaaaactctacacactctctctttctcatgccaaaaatgagtccttccaagacaccccaagtacattgtgatcaattaggaaatgcccaacaaactacaacaagaagattcccacaaccaagaaacgtttctgttgtgaggagcgcacctatataaacctattctctgcctacaaaaaaaaaaacccagtgcagtactgcctcttcttttagctgcactcaacatattaatcattagcaccaatactctccatctatagatttcctttctaatttaggaattcattttaacatatttataacaatattctcttttaaatggtagtaattaggtgtgctaaaatggaattccccagtaccccatttgataattcaaacaactctgaagaaagggaagtaggaagaagaacagataaaaggaagcaaattgatggtgaagttaaagaatacaaatccaagaaccttaaggctgagagaaataggcgtcaaaaacttagcgaaaggcttcttcaattacgctcattggtcccaaacataacaaatatgacaaaagaaaccataatcactgacgccatcacctacattagggagctacaaatgaatgtggacaacctaagtgagcagcttcttgaaatggaagcaactcagggggaggaactggagacaaaaaatgaagagattatcgatactgcagacgagatgggtaaatggggcatagagcctgaagttcaagtggctaacattggcccaactaagctttggataaaaatagtctgccaaaagaaaagaggtggattaactaaactgatggaggcaatgaatgctcttggatttgatataaatgacaccagtgccactgcctctaaaggagctattcttattacttcatctgtggaggtggttagaggtggactaactgaagctaatcgaatcagagagatcttactggagatcatccacggaatctactagaaccagcatgaaaatcccctaaatcttaatagctttagtctcattggagaaatgccagagtacctctaatttttagttatggtgtcaaaaaaaggattcaatcatgttttcaaaatattgtatccaaattgtctgaaaaaagctagtgtgaactcttgtttttgctttcaaatattgatgaaacaacttatcatatgttactaattactctctcctatcttaattctatagcatttcatatgcagtcaagtttggcttaattcaactgaggcaaactttgaaatagaatatactactttttaacttggattttccatggtgggatccaggctccaaattttttggcttctctatatgaaacaatgaaaaaataacatttttggtcccatatccagctgaagaaacagctaaataataaaagccagcacattaagtgtcctttctgtttgaatattaaacaaacaccaatattagacgtgtgaagagtgaaatttatttctacctataatacaacaggacatttgtttcactgtcattaaattcttgttttcttctctgttctttgaccaatgtcgtcctcaatgaagagtcgaaaataagcctgccttaagcatcaccttcccttgtacaggatccaaataataaaagactacttggaaaaccaaaatcagacgtcgaatcgtcaaaatttaataccatggaaagtgtacctacccttcttataatattaacacgaaaaagactctcacaagtctatattaataggactaaaacatgcacacaattgtagcaatattcatattttctatggctaggcttagttttctcctcatttgcctagttatagctcaggttaacttgcttatgcgaaatgctaatgcacttagaaaactagacaccttcacagtagtcccaagtcctgcgccgtttgaaggaggaatggacagaggaagaagtgatcaagctccagcaatcacacatataaggactcatcattctttcaacaagttcatggctggtggagacttgatccttggcggcttcgccactgctctaattgcttccattttttgttacattcgagtcacaggacgaagaaaccaacatagtctgggctgaggcacagcatttttaattttgtactacttttagtttccaaatcaaatatatacacagggttcatgattttgctca

SEQ ID NO: 3 - protein, 230 aa, Solanum lycopersicum
MVVKVYGSAMAACPQRVMVCLIELGVDYELIHVDLDSLQQKKPDFLLLQPFGQVPVIEEGDFRLFESRAIIRYYAAKYEDKGKKLTGTTLEEKALVDQWLEVESNNYNDLVYNMVLQLLVFPKMGHKSDLIVVQKCANNLEKVFDIYEQRLSKSKYLAGDFFSLADLSHLPSLRFLMNEGGFAHLVTQRKYLHDWYLDISSRPSWSKVLDFMNLKKLEMLPGPPKEEVKV

SEQ ID NO: 4 - DNA, 960 bp, Solanum lycopersicum
tatatatatggtcaattattgatcacaaataagaagattattagaaatttcaacctggtgtactaggacatcacaacaaaaaaaaaaagaggaaaaatggtagtgaaagtgtatggttcagcaatggctgcatgtccacaaagggtcatggtttgtcttatagaattgggagtcgattatgaacttatacatgttgatcttgattctctccagcagaaaaaacctgatttcttgcttttacagccatttggacaggttcctgtcattgaagagggcgatttcaggcttttcgaatctagagcaataataaggtactatgcagcaaaatatgaagacaagggaaagaaactaaccggaacgacattggaagaaaaagctctagtagatcaatggctagaagtggaatccaacaactacaatgacttggtatacaacatggtactccaactcctcgtattccctaaaatgggacacaaaagtgacttgatcgtcgtacaaaaatgtgccaacaatttagagaaagtgttcgatatctatgaacaaaggttgtccaagagtaaatacttagcaggagattttttctccttagctgatctaagccacctccctagccttagatttttgatgaatgaaggtggctttgcacatttggtgactcaaaggaagtatttgcatgattggtatttggatatttcaagtaggccttcttggagcaaagtgttggacttcatgaatttgaagaaattagagatgttacccggcccacctaaagaagaagtaaaagtttaacaaacactacaacgccatgattattctgttacgagtggcatggatactgagaattcatattgccaactctgtctatctaatcagttaggttttgaaattgaagcgttttgtgtttcgttgtgttgtaatcaccaaaaataaaataaaattgaactatgggtttaccatcaa

SEQ ID NO: 5 - DNA, 23 bp, Artificial sequence
  Synthetic sequence
ggcgtcaaaaacttagcgaaagg

SEQ ID NO: 6 - DNA, 23 bp, Artificial sequence
  Synthetic sequence
atacaaatccaagaaccttaagg

SEQ ID NO: 7 - DNA, 23 bp, Artificial sequence
  Synthetic sequence
gaaagtgtatggttcagcaatgg

SEQ ID NO: 8 - DNA, 23 bp, Artificial sequence
  Synthetic sequence
caatggctgcatgtccacaaagg

SEQ ID NO: 9 - DNA, 20 bp, Artificial sequence
  Synthetic sequence
tggttgctgctcatcttcac

SEQ ID NO: 10 - DNA, 20 bp, Artificial sequence
  Synthetic sequence
gcaaagccaccttcattcat

SEQ ID NO: 11 - DNA, 20 bp, Artificial sequence
  Synthetic sequence
taggggattttcatgctggt

SEQ ID NO: 12 - DNA, 20 bp, Artificial sequence
  Synthetic sequence
gccaaaaatgagtccttcca

SEQ ID NO: 13 - DNA, 26 bp, Artificial sequence
  Synthetic sequence
actgtccctatctatgaaggttatgc

SEQ ID NO: 14 - DNA, 23 bp, Artificial sequence
  Synthetic sequence
gaaacagacaggacactcgcact

SEQ ID NO: 15 - DNA, 23 bp, Artificial sequence
  Synthetic sequence
aactctgaagaaagggaagtagg

SEQ ID NO: 16 - DNA, 23 bp, Artificial sequence
  Synthetic sequence
attcaaacaactctgaagaaagg

SEQ ID NO: 17 - DNA, 23 bp, Artificial sequence
  Synthetic sequence
aatggctgcatgtccacaaaggg

SEQ ID NO: 18 - DNA, 23 bp, Artificial sequence
  Synthetic sequence
atggtttgtcttatagaattggg

SEQ ID NO: 19 - DNA, 91 bp, Solanum lycopersicum
ccaagaaccttaaggctgagagaaataggcgtcaaaaacttagccaatggctgcatgtccacaaagggtcatggtttgtcttatagaattg

SEQ ID NO: 20 - DNA, 44 bp, Solanum lycopersicum
ccaagaaccttaaggctgagagaaataggcgtcaaaaacttagc

SEQ ID NO: 21 - DNA, 47 bp, Solanum lycopersicum
caatggctgcatgtccacaaagggtcatggtttgtcttatagaattg

SEQ ID NO: 22 - DNA, 89 bp, Solanum lycopersicum
cttcaccatcaatttgcttccttttatctgttcttcttcctactgtggacatgcagccattgctgaaccatacactttcactaccattt

SEQ ID NO: 23 - DNA, 44 bp, Solanum lycopersicum
cttcaccatcaatttgcttccttttatctgttcttcttcctact

SEQ ID NO: 24 - DNA, 45 bp, Solanum lycopersicum
gtggacatgcagccattgctgaaccatacactttcactaccattt

SEQ ID NO: 25 - DNA, 23 bp, Artificial sequence
  Synthetic sequence
cctacttccctttcttcagagtt

SEQ ID NO: 26 - DNA, 23 bp, Artificial sequence
  Synthetic sequence
ccctttgtggacatgcagccatt

SEQ ID NO: 27 - DNA, 86 bp, Solanum lycopersicum
gaattccccagtaccccatttgataattcaaacaactctgaattgggagtcgattatgaacttatacatgttgatcttgattctct

SEQ ID NO: 28 - DNA, 42 bp, Solanum lycopersicum
gaattccccagtaccccatttgataattcaaacaactctgaa

SEQ ID NO: 29 - DNA, 44 bp, Solanum lycopersicum
ttgggagtcgattatgaacttatacatgttgatcttgattctct